(19) United States
(12) Patent Application Publication
(10) Pub. No.: US 2026/0347941 A1
(43) Pub. Date: Nov. 5, 2026

(54) METHODS OF DETECTION AND ANALYSIS OF NUCLEIC ACID IN CIRCULATING BODILY FLUIDS TO DIAGNOSE COGNITIVE DISORDERS

(71) Applicant: BRAIN CHEMISTRY LABS, Jackson, WY (US)

(72) Inventors: Sandra Anne BANACK, Jackson, WY (US); Rachael DUNLOP, Jackson, WY (US); Paul Alan COX, Jackson, WY (US)

(21) Appl. No.: 19/534,424

(22) Filed: Feb. 9, 2026

Related U.S. Application Data
(60) Provisional application No. 63/756,720, filed on Feb. 10, 2025.

(51) Int. Cl. C12Q 1/6883; G16H 50/30

(52) U.S. Cl. CPC C12Q 1/6883; G16H 50/30; C12Q 2600/178

(57) ABSTRACT

Presented herein are methods, systems, and a model useful in identifying a subject who has, or is at risk of developing Alzheimer's Disease (AD) and/or Mild Cognitive Impairment (MCI), or identifying a subject who has or is at risk of progressing from MCI to AD, or increasing in the severity of AD symptoms that includes determining a presence or amount of two or more micro-RNAs (miRNAs) selected from 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p in a subject's circulating blood, without determining a presence or amount of the miRNAs from neural-derived exosomes. Also presented herein are methods of preventing, treating, or delaying the onset of AD and/or MCI.

### FIELD OF THE EMBODIMENTS

Certain embodiments relate to methods of detecting the presence of, the absence of, or the amount of one or more miRNAs in bodily fluids, including circulating blood, and determining the presence or absence of Alzheimer's Disease (AD) and/or Mild Cognitive Impairment (MCI). Certain embodiments also relate to specific miRNAs, or subsets thereof, that are predictive and/or diagnostic of AD and/or MCI, as well as methods of monitoring treatments, methods of treatment, and kits for diagnostic purposes. Other embodiments include a model useful in determining the presence or absence of Alzheimer's Disease (AD) and/or Mild Cognitive Impairment (MCI) that includes a database of miRNA and software configured to analyze miRNA data from a sample to predict the presence or absence of Alzheimer's Disease (AD) and/or Mild Cognitive Impairment (MCI).

### INTRODUCTION

The need for biomarkers for all neurodegenerative diseases is well established. See, ALS Strategic Plan 2023, available on nih.gov; Vignaroli, et al., “The Need for Biomarkers in the ALS-FTD Spectrum: A Clinical Point of View on the Role of Proteomics,” Proteomes 11(1):1-18 (2023). Biomarkers are essential for reducing diagnostic delays and improving disease outcomes as well as for therapeutic drug development. To improve reproducibility and ultimately to achieve biomarker adoption, every biomarker should undergo robust validation (U.S. Department of Health and Human Services. Biomarker Qualification: Evidentiary Framework Guidance for Industry and Staff 2018, available at fda.gov). The most effective biomarkers are reliable measures of disease (or disease state), applicable across drug development trials, and independent of the drug being tested.

Applicant has previously disclosed an application entitled: “Blood Indicators of Alzheimer's Disease,” U.S. Patent Application Publication No. 2022/0252620, the disclosure of which is incorporated by reference herein in its entirety. This prior application discloses methods of detecting the presence, absence or amount of 2-aminoethyl dihydrogen phosphate and/or taurine in a sample. The present disclosure relates to the detection of the presence, absence, or amount of certain miRNA in a bodily fluid sample to determine the presence or absence of Alzheimer's Disease (AD), to monitor the progression of AD, and/or to determine a treatment of AD.

Ninety-five percent of Alzheimer's disease (AD) cases occur in people over 60-65 years old. This is typically a time in life when individuals are looking forward to their golden years and are poised to make significant contributions to their families and communities. AD robs them not only of life, but prior to death burdens them with inability to form or access short-term memories, increasing confusion, disorientation, social withdrawal, and increasing dementia which intensifies during their last years of life. Neuropathologically, onset of AD symptoms are sometimes correlated with brain neuropathology, particularly amyloid-□-42 (A□42) plaques and a dense tauopathy of neurofibrillary tangles (NFTs) composed of hyperphosphorylated tau deposits in specific brain regions.

There is likely a significant latency period between the initiation of AD-type neuropathology in the brain and onset of clinical symptoms. A study using ¹⁴C decay from detonation of the 50 megaton Tsara bomba by the Soviet Union on Oct. 31, 1961, which injected a tremendous amount of ¹⁴C into the atmosphere, allowed precise dating of NFTs and A□142 plaques extracted from postmortem brain tissues from AD patients. In two of the six patients studied, the NFTs were dated seven years prior to onset of symptoms and in one patient the A□42 plaque dated nine years prior to symptom onset. The advantage of discovering and using biomarkers for AD, particularly during the latency period, could be used to explore new types of treatment or even test previously failed drugs, which could eventually be prescribed at a much earlier period of disease progression.

A recent meta-analysis suggested that several CSF biomarkers resulting from amyloid deposition, neuronal damage and loss, and the formation of NFTs, notably phosphorylated-tau (P-tau), amyloid-beta (Aβ42), total-tau (T-tau), as well as neurofilament light protein (NFL), were strongly associated with AD. Blood plasma concentration of T-tau was also indicated as a potentially good indicator of AD, requiring further research, while plasma concentrations of Aβ40 and Aβ42 did not appear to be good candidates for AD diagnosis. Imaging biomarkers assessing amyloid-beta plaques (PiB-PET scans), tau deposits (tau-PET), brain atrophy (structural MRI), memory-related activity patterns (fMRI), and decreased glucose metabolism (FDG-PET) are also currently in use as AD biomarkers.

Current diagnosis of AD remains uncertain (Dubois et al Lancet Neurol. 2014; 13(6):614-29) and it is based on combination of battery of clinical and neuropsychological tests and neuroimaging, such as structural imaging using Magnetic Resonance Imaging (MRI) and/or glucose metabolism using Positron emission tomography (PET) of fluorodeoxyglucose F18 (¹⁸F-FDG), with an accuracy of less than 70%-85% depending on the method and the severity stage of the disease (Frisoni et al., Nat Rev Neurol. 2010; 6(2):67-77; Tahmasian et al., J Nucl Med. 2016; 57(3):410-5. More recently a plasma analyses by mass spectrometry is being used to determine the ratio of plasma phosphorylated tau 217 (p-tau217) to non-p-tau217 and a ratio of amyloid-β 42 and amyloid-β 40 (Palmqvist et al. 2024).

Some research has been conducted on the use of micro RNA (miRNA) and long non-coding RNA (lncRNA) as diagnostic biomarkers for AD because they are known to circulate and tissue specific profiles can be identified in body fluids such as serum, plasma, CSF, and urine. U.S. Pat. No. 11,718,879 claims a method of detecting levels of lncRNAs in a lncRNA signature that includes 13 lncRNAs of Inc-DLGS5-1:1, Inc-EBLN1-1:4, Inc-FAT1-7:2, Inc-PRR5-5:1, Inc-RBKS-6:1, Inc-FOXD4L5-35:1, Inc-TENM3-3:3, Inc-FAM133B-2:1, Inc-ZNF726-1:3, Inc-AP3M1-1:1, Inc-DUSP10-6:1, Inc-TPPP-1:2, and LINC01206:20. This patent discloses a number of additional signatures including miRNA signatures that include miR.99a.5p, and signatures of 3, 7, 11, 15, 28, and 74 miRNAs.

Others have disclosed measuring levels of or determining the presence or absence of miRNA in fluids to diagnose various neurodegenerative diseases. For example, U.S. Pat. No. 8,648,017 claims measuring levels of normalizer small RNA not expressed in the brain and selected from miR-181a, miR-491-5p, miR-10b, and miR-141. US Patent Application Publication No. 2023/0041359 discloses diagnosing a disease or disorder by measuring in a biological sample one or more miRNAs selected from over 47,972 miRNAs.

Exosomes have been shown to comprise nucleic acids, including messenger RNA (mRNA), microRNA (miRNA), long non-coding RNA (siRNA), tRNA-derived small RNA (tsRNA), and circRNA, which can be transferred from one cell to another and can accumulate in circulating blood and cerebrospinal fluid. Exosomes that are released into the extracellular matrix and taken up by adjacent cells can potentially transfer information from one cell to another. Such information can be therapeutic or pathogenic. Detecting and analyzing miRNAs associated with neural-derived exosomes (or neural-enriched extracellular vesicles (NEE), and use of such detection and analysis in diagnosing and early detection of cognitive disorders, such as ALS, AD, and others, is described in U.S. Patent Application Publication No. 2021/0164051, the disclosure of which is incorporated herein by reference in its entirety.

As described therein, neural-derived exosome fractions, and/or fractions comprising portions or components of exosomes of neural cell or neural tissue origin (individually and collectively referred to interchangeably as “a neural-derived exosome fraction” or “neural-enriched exosome fractions” or “neural-enriched extracellular vesicles”), can be detected, isolated, enriched, or prepared from a sample, for example, a sample obtained from a subject. Thus, a sample may be obtained from a subject, neural derived exosomes, if detected, are isolated and enriched, and then the exosomes are analyzed for the presence, absence, and/or amount of miRNAs present in the neural-derived exosomes. While these methods are useful in early diagnoses and early detection of cognitive disorders, the method requires at least the isolation of neural derived exosomes from the sample. The use of neural-derived exosomes also is useful in determining correlations between one or more miRNA, typically 2 or 3, and the presence of indication of a neurodegenerative disorder, such that the presence, absence, and/or amount of a group of miRNAs selected using neural-derived exosomes can be used to diagnose, monitor, and assess treatment efficacy for certain neurodegenerative disorders. The inventors did not heretofore believe that analyzing circulating bodily fluids for the presence, absence, and/or amount of miRNAs without exosome isolation would or could provide an accurate detection or diagnosis of cognitive disorders.

The first symptomatic stage of AD that is manifested by mild clinical symptoms is Mild Cognitive Impairment (MCI), which is usually defined as an intermediate state between normal aging and dementia (DeCarli, Lancet Neurol., 2003, 2:15-21; Stephan et al., Alzheimer's Res Therapy, 2009, 1:1-9; Apostolova et al., Human Brain Mapping, 2010, 31:786-797). On average, MCI patients convert to dementia at a rate of 10-15% annually (Petersen et al., Arch Neurol. 2001, 58:1985-1992; Apostolova et al., Human Brain Mapping, 2010, 31:786-797). However, currently the MCI outcome is not reliably predictable. First, up to 40% of MCI patients revert to normal status (Larrieu et al., Neurology, 2002, 59:1594-1599; Brooks, Loewenstein, Alzheimer's Res Therapy, 2010, 2:28-36), and autopsy studies demonstrate that a substantial percentage of MCI patients do not have evidence of AD pathology (Jicha et al., Arch Neurol, 2006, 63:674-681; Khan, Alkon, Neurobiol. Aging, 2010, 31:889-900). Second, about 20% of MCI patients who convert to dementia are diagnosed not with AD but other neurodegenerative diseases, such as vascular, Lewy body, Huntington, Parkinson, and other dementias (Jicha et al., Arch Neurol, 2006, 63:674-681; Stephan et al., Alzheimer's Res Therapy, 2009, 1:1-9). Third, disease progression varies for AD patients from slow to intermediate and rapid (Doody et al., Alzheimer's Res Therapy, 2010, 2:2-10). Even clinically MCI is not a homogeneous pathology and can be described as two conditions, with amnestic symptoms (aMCI) and without amnestic symptoms (Dlugaj et al., Dement Geriatr Cogn Disord., 2010, 30:362-373; Brooks, Loewenstein, Alzheimer's Res Therapy, 2010, 2:28-36). Some publications have demonstrated that aMCI converts to dementia much more often and is a better predictor of AD (Mariani et al., J Alzheimer's Dis., 2007, 12:23-35; Luck et al., Psychiatr Prax., 2008, 35:331-336; Koivunen et al., Neurology, 2011, 76:1085-1099). However, other authors have not found significant difference in the conversion rate for two MCI forms (Rountree et al., Dement Geriatr Cogn Disord., 2007, 24:476-482).

While some of the afore-discussed subject matter is discussed with reference to known literature and publications, the embodiments described herein may include one or more known features, without suffering from drawbacks and/or problems previously encountered. In addition, information presented in this Introduction section is not an admission that the information is prior art to the embodiments described herein, expressly including the preceding paragraph.

### SUMMARY

Presented herein, in certain embodiments, are methods of detection and analysis of miRNAs in circulating bodily fluids without exosome isolation. In some embodiments, such methods can be used for the diagnosis and early detection of a variety of cognitive disorders and disorders, including Alzheimer's Disease (AD) and/or Mild Cognitive Impairment (MCI). Also presented herein, in certain embodiments, are methods of determining the relationship between the types and amounts of miRNAs and the absence and/or presence of one or more neurological diseases or disorders, and then using that relationship in a method for the diagnosis and early detection of one or more neurological diseases or disorders by detection and analysis of miRNAs in circulating bodily fluids without exosome isolation.

In some aspects, presented herein is method of identifying a subject who has, is at risk of developing a cognitive disorder, or is being tested to exclude a possible future AD and/or MCI diagnosis comprising: (a) determining a presence or amount of one or more micro-RNAs (miRNAs) in a sample obtained from the subject without determining the presence or amount in neural derived exosomes, wherein the one or more miRNA are selected from the group consisting of at least 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p, and (b) determining if the subject has, or is at risk of developing AD and/or MCI according to the presence or amount of the one or more miRNAs in the sample.

In some aspects, presented herein is a method of preventing or treating a cognitive disorder in a subject who has, or is at risk of developing AD and/or MCI, the method comprising: (a) determining a presence or amount of one or more micro-RNAs (miRNAs) in a sample obtained from the subject without determining the presence or amount in neural derived exosomes, wherein the one or more miRNA are selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p; (b) determining if the subject has, or is at risk of developing AD and/or MCI according to the presence or amount of the one or more miRNAs in the sample; and (c) administering an AD and/or MCI treatment to the subject when the determining of (b) determines that the subject has, or is at risk of developing AD and/or MCI. In some embodiments, a cognitive disorder treatment comprises administering a therapeutically effective amount of L-serine, and the cognitive disorder is AD and/or MCI.

In some aspects, presented herein is one or more kits useful in carrying out a method of identifying a subject who has, or is at risk of developing AD and/or MCI that includes one or more multi-well plates, with each well containing one or more miRNA primers selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p, and a mechanism to calculate the likelihood of a positive or negative diagnosis, and/or probability of having or not having AD and/or MCI. In one embodiment, the kit includes two well-plates in which one well plate has each well containing one or more miRNA primers selected from the list above, and the other well plate includes a Quality Control (QC)/spike-in/calibration plate in which one or more wells contain QC primers, no-template controls, and/or spike-in primers designed to monitor the success of the method. In another embodiment, the kit includes one well-plate in which the wells include one or more miRNA primers selected from the list above, QC primers, no-template controls, and/or spike-in primers designed to monitor the success of the method. In another embodiment, the kit includes one well-plate in which the wells include one or more miRNA primers selected from the list above. In a further embodiment, the mechanism to calculate the likelihood of a positive or negative diagnosis, and/or probability of having or not having AD and/or MCI includes software configured to carry out the calculation using random forest machine learning algorithms and/or logistic regression algorithms developed from machine learning classification of known AD and/or MCI and healthy control plasma samples.

Other aspects of the technology include a model and/or system useful in detecting the presence or absence of AD and/or MCI. The model and/or system may include at least: (1) a database stored in a storage medium, wherein the database comprises dCq values for individuals with a known disease status, a list of miRNA and a coefficient associated with each miRNA, wherein the list of miRNA is selected from 4 or more miRNA selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p; and (2) software configured to access the database, process qPCR generated quantification data for each miRNA in the list; generate normalized qPCR values for each miRNA in the list, and process the normalized qPCR values for each miRNA by computing and outputting a predicted diagnosis of AD and/or MCI using the software. The predictive certainty uses parameters pre-set into the software to provide the computation. Additional aspects of the embodiments include methods of developing the model and creating the software configured to carry out the processing steps above.

Certain aspects of the technology are described further in the following description, examples, and claims.

### DETAILED DESCRIPTION

As described herein, the miRNA content of circulating bodily fluids has diagnostic and therapeutic utility. The identification of the specific miRNA useful in detecting certain neurodegenerative disorders, or the risk of developing certain neurodegenerative disorders, such as AD and/or MCI can be determined by methods disclosed in, for example, U.S. Patent Application Publication No. 2021/0164051, entitled: “Methods of Detection and Analysis of Nucleic Acid in Neural-Derived Exosomes,” the disclosure of which is incorporated by reference herein in its entirety. While the use of exosomes derived from neural cells is useful in the initial identification of miRNA and analyzing the relationships between healthy and un-healthy individuals, using exosomes derived from neural cells in a diagnostic method is complex and cumbersome. The inventors discovered, quite unexpectedly, that detecting the onset or progression or risk of developing a neurodegenerative disorder by determining a presence or amount of one or more micro-RNAs (miRNAs) in a circulating bodily fluid sample (e.g., blood) without isolating and assessing the presence or amount of one or more miRNAs in neural-derived exosomes, was more accurate than determining a presence or amount of one or more miRNAs in exosomes derived from neural cells. Thus, the diagnostic methods described herein are not only simpler and easier than using exosomes derived from neuronal cells, but they are unexpectedly more accurate and predictive.

As discussed above, the techniques described in U.S. Patent Application Publication No. 2021/0164051, can be useful in determining the one or more miRNAs that could be used to identify a subject who has, or is at risk of developing a cognitive disorder. The abundance of, for example, tetraspanins and cell adhesion molecules (CAMs) expressed on or in exosomes derived from neural cells or neural tissues makes it possible to detect, enrich, prepare, and/or isolate neural-derived exosomes. Such neural-derived exosomes may be employed to detect and/or quantify the amount of certain exosome-derived miRNAs. The presence or amount of certain exosome-derived miRNAs, or sets of such miRNAs, provides insight as to whether a subject has, or is at risk of developing certain cognitive disorders. For example, the presence or amount of certain exosome-derived miRNAs can also be used to provide early diagnosis of a cognitive disorder and/or to identify subjects who are at risk of developing a cognitive disorder. Once one or more miRNAs have been identified using exosomes as being useful in identifying a subject who has or is at risk of developing a cognitive disorder, using the guidelines provided herein, the one or more miRNAs so identified, then can be detected (presence and/or amount) in circulating bodily fluids such as blood without isolating exosomes, and the presence or amount of the miRNAs used to identify such subjects. Such diagnostic methods are far simpler to carry out using circulating bodily fluids, and can be more accurate, when compared to using exosomes derived from neuronal cells.

Alzheimer's Disease (AD) and Mild Cognitive Impairment (MCI) are non-limiting examples of cognitive disorders, and are a form of a cognitive disorder. There are a variety of genetic and environmental risk factors that may lead to AD and/or MCI which are believed to result from gene/environment interactions and these disorders likely represent a syndrome rather than a single disease. The onset of AD and/or MCI symptoms often presents a crisis for patients and their families.

One of the problems in current AD and/or MCI therapy is difficulty of diagnosis. In its initial presentation, AD and/or MCI are often misdiagnosed. Clinical diagnosis of AD and/or MCI are difficult because symptoms overlap with other types of dementia, some of which have available treatments. Furthermore, accurate diagnosis of dementia related disorders are based on the underlying neuropathology, which is difficult to determine in living patients. This precious time lost due to the inability to diagnose presents a significant burden on patients and their families, as well as their physicians, because of the inability to prescribe medication or even to plan for treatment and patient care.

In one embodiment, there is provided an AD and/or MCI diagnostic multi-miRNA fingerprint (one or more, two or more, three or more, four or more, five or more, up to all of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p, obtained from a standard clinical blood draw that may be useful in diagnosing AD and/or MCI, determining subjects at risk of developing AD and/or MCI, testing subject to exclude a future AD and/or MCI diagnosis, and in assessing therapies for AD and/or MCI. miRNA were identified from an earlier experiment on blood plasma from neural enriched extracellular vesicles in which the miRNA were shown by next generation sequencing to be differentially expressed between AD and/or MCI patients and healthy controls. The bilipid membrane of the extracellular vesicles contributed to reproducibility in two ways: 1) protecting miRNA from degradation, and 2) facilitating purification of the sample by immunopurification through transmembrane proteins on the surface of the extracellular vesicles. Dunlop R A, et al., “L1CAM immunocapture generates a unique extracellular vesicle population with a reproducible miRNA fingerprint,” RNA biology, 20(1):140-148 (2023).

The identified miRNA fingerprint fills an unmet AD and/or MCI drug development and medical diagnostic need and could facilitate the identification of AD and/or MCI, or subjects at risk of developing AD and/or MCI, at its earliest stages, thereby reducing diagnostic uncertainty. The inventors surprisingly discovered that the miRNA fingerprint also was found in circulating blood, without exosome isolation, purification, and analysis, and that the method of diagnosing AD and/or MCI, identifying subjects at risk of developing AD and/or MCI, or testing a subject to exclude a future diagnosis of AD and/or MCI, using this miRNA fingerprint from a standard clinical blood draw was more accurate than prior identification techniques that relied on isolation and analysis of miRNA present within neural-enriched extracellular vesicles. The embodiments described herein present data from patients with AD and/or MCI along with control data from healthy individuals.

A “sample” or “samples”, as used interchangeable herein, is often obtained from a suitable subject. A sample can be isolated or obtained directly from a subject or part thereof. In some embodiments, a sample obtained from a subject is a sample derived from the subject. Accordingly, in certain embodiments, a sample obtained from a subject is a sample obtained directly from the subject. In certain embodiments, a sample obtained from a subject is obtained from a third party, for example a third party who obtained or extracted the sample from the subject. In some embodiments, a sample is obtained indirectly from an individual or a medical professional. A sample can be any specimen that is isolated or obtained from a subject or part thereof. Non-limiting examples of samples include fluids or tissues obtained or derived from a subject, including, without limitation, circulating bodily fluids such as blood or a blood product (e.g., serum, plasma, platelets, buffy coats, lymphatic fluid or the like), umbilical cord blood, chorionic villi, amniotic fluid, cerebrospinal fluid (CSF), spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells, neurons) or parts thereof (e.g., mitochondrial, nucleus, extracts, lysates, or the like), urine, feces, sputum, saliva, nasal mucous, prostate fluid, lavage, semen, lymphatic fluid, bile, tears, sweat, breast milk, breast fluid, the like or combinations thereof. In an embodiment, a “sample” is blood.

As used herein, the expressions “circulating bodily fluid” or “circulating body fluid” denotes samples, as described above, that circulate throughout the body. Particularly preferred circulating bodily fluids include, but are not limited to, blood, serum, plasma, platelets, buffy coats, lymphatic fluid, urine, semen, spinal fluid, bile, and the like. Blood is especially preferred due to its ease of sampling.

Non-limiting examples of subjects include mammals, humans, non-human primates (e.g., apes, gibbons, chimpanzees, orangutans, monkeys, macaques, and the like), domestic animals (e.g., dogs and cats), farm animals (e.g., horses, cows, goats, sheep, and pigs) and experimental animals (e.g., mouse, rat, rabbit, and guinea pig). In some embodiments a subject is a mammal. A mammal can be any age or at any stage of development (e.g., an adult (e.g., 18, 19, 20 or 21 years and older), a senior adult (e.g., over the age of 55, over the age of 60, or over the age of 65 years), a teen (e.g., age 12 to 19 yrs.), child (e.g., age 1 to 12 yrs.), infant (e.g., from birth to 1 yr.), or a mammal in utero). A mammal can be male or female. In some embodiments, a subject is a human.

In some embodiments, a subject has, is suspected of having, or is at risk of developing, a cognitive disorder. In some embodiments the cognitive disorder is Alzheimer's Disease (AD). In other embodiment, the cognitive disorder is Mild Cognitive Impairment (MCI). In some embodiments, a subject who has a cognitive disorder is a subject diagnosed as having a cognitive disorder by a medical professional (e.g., a medical doctor) based on, for example the presence of one or more diagnostic symptoms and/or the results of one or more standardized diagnostic test results. A subject suspected of having a cognitive disorder is a subject not yet diagnosed as having cognitive dysfunction by a medical professional. In some embodiments, a subject suspected of having a cognitive disorder may display one or more symptoms of cognitive dysfunction, which symptoms are not conclusive evidence that the subject has a cognitive disorder. In some embodiments, a subject who is suspected of a having a cognitive dysfunction may have one or more symptoms of a cognitive disorder, but is not diagnosed as having any one particular cognitive disorder disease because there is not enough data to indicate conclusively that the subject has a particular cognitive disorder disease. In some embodiment, a subject who is suspected of having cognitive disorder is a subject suspected of having AD and/or MCI, but is not diagnosed as having AD and/or MCI by a medical professional.

In some embodiments, a subject is determined to be a subject at risk of developing AD and/or MCI by carrying out one or more of the methods described herein. In some embodiments, a subject at risk of developing AD and/or MCI is a subject who is asymptomatic for AD and/or MCI. In some embodiments, a subject at risk of developing AD and/or MCI is a subject having one or more symptoms of AD and/or MCI, which symptoms may be mild or transient in nature. In some embodiments, a subject at risk of developing AD and/or MCI is a subject who is not yet diagnosed as having AD and/or MCI. In some embodiments, a subject at risk of developing AD and/or MCI is a subject suspected of having AD and/or MCI. In additional embodiments, a subject is a healthy individual tested as a preventative screen to rule out the possibility of being at risk of developing AD and/or MCI and to identify the possibility of onset at an early stage before symptoms arise.

In certain embodiments, the presence or absence of AD and/or MCI in a subject is determined by the methods described herein. In some embodiments, wherein a subject is determined to have AD and/or MCI that is diagnosed by one or more of the methods described herein, there is further provided a method of treating AD and/or MCI by administering a suitable treatment to the subject, wherein the AD and/or MCI, or one or more symptoms thereof are therapeutically treated. In certain embodiments, the methods described herein identify a subject who is at risk of developing AD and/or MCI. In some embodiments, wherein a subject is identified as at risk of developing AD and/or MCI by the methods described herein, there is further provided a method of treating that subject at risk of developing AD and/or MCI by administering a suitable treatment to the subject, wherein development of AD and/or MCI is prevented or delayed, or wherein one or more symptoms thereof are therapeutically treated. In some embodiments, a method of treating AD and/or MCI is a method of inhibiting or delaying the onset or progression of AD and/or MCI, for example in a subject at risk of developing AD and/or MCI. In some embodiments, a method comprises treating AD and/or MCI or one or more symptoms thereof. In some embodiments, a method of treating AD and/or MCI is a method of inhibiting or delaying the onset or progression of one or more symptoms of AD and/or MCI, for example in a subject at risk of developing AD and/or MCI.

Non-limiting examples of a symptom of a cognitive disorder include inability to form new memories; decrease in executive function; inability to manage finances or using numbers; difficulty making decisions; mood swings; inappropriate emotional control; fatigue (e.g., excessive fatigue); passivity; lethargy; loss of motivation; writing difficulty; reading difficulty; difficult thinking; difficulty remembering; difficulty speaking or finding the correct word; withdrawing from social interaction; shortened attention span; difficulty concentrating or maintaining focus; difficulty following directions or following instructions; difficulty solving problems; movement difficulties; unaware of surroundings; the like and combinations thereof. These signs can range from mild to severe. Symptoms may be concomitant with other disorders such as, but not limited to, Parkinson's disease, amyotrophic lateral sclerosis, cardiovascular disease, kidney disease, strokes, diabetes, gastrointestinal disorder, or cancer.

In some embodiments, a method comprises preventing or treating AD and/or MCI, inhibiting or delaying the onset of, or progression of AD and/or MCI, or inhibiting, mitigating, reducing or delaying the onset of one or more symptoms of AD and/or MCI, where the method comprises administering a therapeutically effective amount of a AD and/or MCI disease drug, non-limiting examples of which include L-serine, a cholinesterase inhibitor (e.g., donepezil, galantamine, aricept, or rivastigmine), Memantine, Namzaric, an antidepressant (eg. trazodone), an N-methyl D-aspartate (NMDA) antagonist, an omega-3 fatty acid, curcumin, or a curcumin derivative, vitamin E, vitamin B complex or individual vitamin B's; vitamin D, CoQ10, a sleep aid (e.g., zolpidem, eszopiclone or zaleplon), an anti-anxiety drug (e.g., lorazepam, clonazepam, bupropion, Mirtazapine), an anti-convulsant (e.g., sodium valproate, carbamazepine, or oxcarbazepine), an anti-psychotic (e.g., risperidone, quetiapine or olanzapine), carbidopa-levodopa, amantadine, a dopamine agonists (e.g., pramipexole, ropinirole, rotigotine or Apomorphine), a MAO B inhibitor (e.g., selegiline, rasagiline or safinamide), a SSRI drug (eg. Lexapro, Escitalopram),a Catechol O-methyltransferase (COMT) inhibitors (e.g., entacapone or tolcapone), an anticholinerigic (e.g., benztropine or trihexyphenidyl), immunotherapy, anti-amyloid drugs (eg., Lecanemab-irmb, Leqembi, Donanemab, Aducanumab, Aduhelm, Kisunla), anti-Tau drugs (eg., Leuco-methylthioninium-bis(hydromethanesulfonate), Hydromethylthionine mesylate, Salsalate, Gosuranemab, Ponezumab), Prevagen, Gabapentin, Ocuvite, Atenolol, Namenda, anti-hypertension drug (eg., Valsartan, Benazepril, Losartan, Metoprolol), Blood thinners (eg., Eliquis, Xarelto) the like and combinations thereof. In some embodiments, a subject diagnosed as having AD and/or MCI, or at risk of developing AD and/or MCI is treated by a method comprising administering a therapeutically effective amount of L-serine In some embodiments, a subject diagnosed as having AD and/or MCI, or at risk of developing AD and/or MCI is treated by a method comprising administering a therapeutically effective amount of L-serine.

### L-Serine

In some embodiments, a subject is administered a therapeutically effective amount of L-serine, a salt, metabolic precursor, derivative or conjugate thereof. In some embodiments, a subject is administered a therapeutically effective amount of free L-serine, or a salt thereof. A therapeutically effective amount of L-serine or free L-serine may be administered as a pharmaceutical composition comprising one or more pharmaceutical excipients, additives, carriers and/or diluents. In some embodiments, a method herein comprises administered a therapeutically effective amount of a composition comprising, consisting of, or consisting essentially of L-serine, a salt, metabolic precursor, derivative or conjugate thereof to a subject. In some embodiments, a method herein comprises administered a therapeutically effective amount of a composition comprising, consisting of, or consisting essentially of free L-serine, or a salt, derivative or conjugate thereof to a subject. In some embodiments, a method herein comprises administering a therapeutically effective amount of a composition comprising, consisting of, or consisting essentially of a polymer of L-serine, or a salt, derivative or conjugate thereof to a subject. In some embodiments, a composition consisting essentially of L-serine, free L-serine, or a salt, a precursor, a derivative or a conjugate thereof excludes proteins or protein fractions comprising less than 100%, 99%, 98%, less than 95%, less than 90%, less than 80%, less than 70%, less than 60%, or less than 50% L-serine (wt/wt). In some embodiments, a composition consisting essentially of L-serine, free L-serine, or a salt, a precursor, a derivative or a conjugate thereof excludes proteins or protein fractions comprising greater than 5%, greater than 10%, greater than 20%, greater than 30%, greater than 40%, greater than 50% or greater than 60% protein (wt/wt), and excludes other components that materially affect the therapeutic efficacy of the L-serine to prevent and/or treat AD and/or MCI. In some embodiments, a composition consisting essentially of L-serine comprises free L-serine, or a polymer of L-serine having an amino acid content of L-serine of at least 100%, 99%, 98%, 95%, 90%, 85% or at least 80%. In some embodiments, a composition consisting essentially of L-serine excludes creatine, creatine pyruvate, guanidino-acetic acid (GA), glycocyamine, N-amidinoglycine, and salts or esters thereof. In some embodiments, a composition consisting essentially of L-serine is a composition comprising free L-serine at a purity of at least 85%, at least 90%, at least 95%, at least 98%, at least 99% or 100%. In certain embodiments, a composition consisting essentially of L-serine, free L-serine, or a salt, a precursor, derivative or conjugate of L-serine, is a composition that also comprises zinc.

Free L-serine refers to L-serine in the form of a single amino acid monomer, or a salt thereof. In some embodiments, a composition comprises free L-serine at a purity of at least 85%, at least 90%, at least 95%, at least 98%, at least 99% or 100%. In certain embodiments, free L-serine is not covalently bonded to any other amino acid.

In some embodiments, a composition comprising L-serine may exclude other active ingredients. In some embodiments, a composition may exclude proteins containing L-serine. In some embodiments, a composition may exclude proteins having a molecular weight greater than 10 kDa, greater than 20 kDa, greater than 30 kDa or greater than 50 kDa. In some embodiments, a composition may exclude proteins containing less than 99%, 98%, 95%, 92%, 90%, 80%, 70%, 60%, or less than 50% L-serine. In some embodiments, a composition may exclude creatine, or any energy metabolism precursor of creatine, such as guanidino-acetic acid (GA), equivalents thereof, and mixtures thereof.

In certain embodiments, a composition comprises L-serine, non-limiting examples of which include free L-serine, and polymers or polypeptides comprising at least a 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% L-serine by weight or amino acid content. In some embodiments, a polymer of L-serine or a polypeptide comprising L-serine includes between 2 and 50000, between 2 and 500, between 2 and 100, between 2 and 50, between 2 and 20, between 2 and 15, between 2 and 10, between 2 and 9, between 2 and 8, between 2 and 7, between 2 and 6, between 2 and 5, or between 2 and 4 L-serine amino acids linked by covalent bonds. In certain embodiments, a composition comprises L-serine, non-limiting examples of which include a polymer or polypeptide comprising from 20% to 100%, from 30% to 100%, from 35% to 100%, from 40% to 100%, from 45% to 100%, from 50% to 100%, from 55% to 100%, from 60% to 100%, from 65% to 100%, from 70% to 100%, from 75% to 100%, from 80% to 100%, from 85% to 100%, from 90% to 100%, from 95% to 100%, from 96% to 100%, from 97% to 100%, from 98% to 100%, or from 99% to 100% content of L-serine (wt/wt) or amino acid content (i.e., L-serine monomers/total amino acid monomers).

Non-limiting examples of a salt of L-serine include a sodium salt, potassium salt, calcium salt, magnesium salt, zinc salt, ammonium salt; inorganic salts such as, hydrogen chloride, sodium chloride, potassium chloride, calcium chloride, sodium phosphate, potassium phosphate, and sodium hydrogen carbonate; organic salts such as, sodium citrate, citrate, acetate, and the like. In certain embodiments, a composition comprises L-serine as an alkylated L-serine, such as L-serine with an alkyl group, or e.g., an alkyl comprising 1-20 carbon atoms. In certain embodiments, a derivative of L-serine includes an L-serine ester, an L-serine di-ester, a phosphate ester of L-serine, or a sulfate or sulfonate ester of L-serine. Non-limiting examples of a conjugate of L-serine includes a pegylated L-serine (e.g., an L-serine comprising one or more polyethylene glycol (PEG) moieties), and a lipidated L-serine. Non-limiting example of a precursor of L-serine include L-phosphoserine.

Non-limiting examples of a precursor of L-serine include a pro-form of L-serine that is broken down into L-serine monomers by the digestive system of a subject. In some embodiments, L-serine or a conjugate thereof consists of a slow-release version. In some embodiments a derivative of L-serine is conjugated to a different molecule forming a prodrug from which L-serine is released after crossing the blood/brain barrier.

In some embodiments, a composition consisting essentially of L-serine may comprise some amount of D-serine. For example, a composition consisting essentially of L-serine may include a small amount of D-serine, for example, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, or less than 0.1% D-serine by weight (e.g., wt/wt) or amino acid content (e.g., L-serine/total amino acid content). For example, a composition may include from 0.001% to 30%, from 0.005% to 30%, from 0.1% to 30%, from 1% to 30%, from 2% to 30%, from 3% to 30%, from 4% to 30%, from 5% to 30%, from 6% to 30%, from 7% to 30%, from 8% to 30%, from 9% to 30%, from 10% to 30%, from 0.001% to 20%, from 0.005% to 0%, from 0.1% to 20%, from 1% to 20%, from 2% to 20%, from 3% to 20%, from 4% to 20%, from 5% to 20%, from 6% to 20%, from 7% to 20%, from 8% to 20%, from 9% to 20%, or from 10% to 20% D-serine. In some embodiments, a composition comprising or consisting essentially of L-serine, does not comprise a substantial amount of D-serine. In some embodiments, a composition comprising or consisting essentially of L-serine, does not contain D-serine.

### Methods Using Circulating Bodily Fluid

In some embodiments, a method presented herein detects or determines an amount of one or more miRNAs associated with AD and/or MCI from circulating bodily fluid, for example, from a subject's blood, without isolating exosomes. The inventors' prior efforts carried out methods using exosomes, where the isolation, separation, identification, etc. of various miRNAs can be time consuming and cumbersome. While the previous method was effective in identifying panels of miRNAs associated with a cognitive disorder such as AD and/or MCI, using exosomes as a commercial diagnostic poses some difficulties, including difficulty in automation, labor and time intensity, etc. It also would not have been known or appreciated that the same or all of miRNAs identified using neural-derived exosomes could also be detected in a subject's circulating body fluid without isolating and analyzing exosomes, nor would it have been expected that a diagnostic test using the same or all of these miRNAs from circulating bodily fluid would be as accurate or sensitive as a diagnostic test using neural-derived exosomes.

The inventors unexpectedly discovered that the same miRNA can be found in circulating blood plasma without the neural-enriched or derived extracellular vesicles (NEE, exosomes) isolation and analysis steps, and that the same miRNA are more concentrated, providing a better signal for diagnostic applications. In some embodiments, a method herein comprises determining the presence or amount of one or more miRNAs selected from one or more of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p from a subject's circulating bodily fluid. In some embodiments, a method herein comprises determining the presence or amount of one or more miRNAs selected from one or more of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p from a subject's circulating bodily fluid prepared from a sample obtained from a subject that has, or is suspected of having, a cognitive disorder. In some embodiments, a method herein comprises determining the presence or amount of one or more miRNAs selected from one or more of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p from a subject's circulating bodily fluid prepared from a sample obtained from a control subject. Non-limiting examples of a control subject include healthy subjects, a subject that does not have a cognitive disorder and/or a subject that is not suspected of having a cognitive disorder.

A feature of an embodiment includes a method of identifying a subject who has, or is at risk of developing a cognitive disorder such as AD and/or MCI, the method comprising: (a) determining a presence or amount of one or more micro-RNAs (miRNAs) selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p from a subject's circulating bodily fluid without determining a presence or amount from a subject's neural-derived exosomes, and determining if the subject has, or is at risk of developing the cognitive disorder such as AD and/or MCI, according to the presence or amount of the one or more miRNAs in the sample. In certain embodiments, the method comprises determining the presence or amount of two or more, three or more, four or more, five or more, six or more, seven or more, eight or more, nine or more, ten or more, on up to all 43 of of the miRNAs selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p. In certain embodiments, the presence of four or more, five or more, six or more, seven or more or eight or more of the miRNAs selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p in, on or within a subject's circulating bodily fluid obtained from a subject, indicates that the subject has or is at risk of having a cognitive disorder such as AD and/or MCI.

In some embodiments, a method herein comprises comparing an amount of one or more miRNAs in circulating bodily fluid obtained from a sample from a control subject (e.g., a subject known to be free of a cognitive disorder) to an amount of one or more miRNAs in circulating bodily fluid obtained from a sample from a test subject (e.g., a subject suspected of having a cognitive disorder). In some embodiments, the presence or absence of a cognitive disorder in a test subject is determined according to such a comparison. In some embodiments, a subject at risk of developing a cognitive disorder is identified according to such a comparison. In some embodiments, a comparison determines that the amount of one or more miRNAs associated with neural-derived exosomes obtained from a first (or test) subject are significantly lower, or significantly higher than those obtained from a control subject.

The term “significantly” as used throughout refers to a statically significant difference that can be determined using a suitable statistical method (e.g., a t-test). In some embodiments, a comparison determines that the amount of one or more miRNAs obtained from a sample of a first subject's circulating bodily fluid are significantly higher than those of a control subject, thereby indicating that the first subject has a neurodegenerative disease or has a high statistical likelihood of developing a neurodegenerative disease.

In some embodiments, a comparison determines that the amount of one or more miRNAs obtained from a sample of a subject's circulating bodily fluid is from about 1.1-fold to about 50 fold higher or lower than a baseline amount of such one or more miRNAs, thereby indicating that the subject has a neurodegenerative disease or has a statistical likelihood of developing a neurodegenerative disease (i.e., is “at risk” of developing, e.g., a cognitive disorder such as, e.g., AD and/or MCI). In some embodiments, a comparison determines that the amount of one or more miRNAs obtained from a sample of a first subject's circulating bodily fluid is from about 1.5 to about 50 fold higher or lower, or any value or ranges of values therebetween, than the baseline amount of such one or more miRNAs, thereby indicating that the subject has a neurodegenerative disease or has a statistical likelihood of developing a neurodegenerative disease (i.e., is “at risk” of developing, e.g., a cognitive disorder such as, e.g., AD and/or MCI). Throughout this description, the disclosure of range, such as from about 5 to about 10, includes any value between 5 and 10, as well as any range of values between 5 and 10 such as 5-9, or 6-10, or 6-9, or 5-8, or 5, 6, 7, 8, 9, or 10, etc.

In some embodiments, an amount of, for example miR-151a-3p that is at least 1.4, at least 1.5, at least 1.6, at least 1.7, at least 1.8, or at least 1.9 fold higher than a base line amount of miR-151a-3p in circulating body fluid obtained from a healthy subject means that the subject has or is at risk of developing AD. In some embodiments, an amount of miR-151a-5p that is at least 0.9, at least 1.0, at least 1.1, or at least 1.2 fold higher than a base line amount of miR-151a-5p in circulating body fluid obtained from a healthy subject means that subject has or is at risk of developing AD. In some embodiments, an amount of miR-146a-5p that is at least 1.2, at least 1.3, at least 1.4, or at least 1.45 fold higher than a base line amount of miR-146a-5p in circulating body fluid obtained from a healthy subject means that subject has or is at risk of developing AD.

In some embodiments, an amount of miR-4454 that is at least 2.0, at least 2.1, at least 2.2, or at least 2.3 fold lower than a base line amount of miR-4454 in in circulating body fluid obtained from a healthy subject means that subject has or is at risk of developing AD. In some embodiments, an amount of miR-10b-5p that is at least 2.9, at least 3.0, at least 3.1, or at least 3.3 fold lower than a base line amount of miR-10b-5p in circulating body fluid obtained from a healthy subject means that subject has or is at risk of developing AD. In some embodiments, an amount of miR-199a-3p that is at least 1.3, at least 1.4, at least 1.5, or at least 1.6 fold higher than a base line amount of miR-199a-3p in circulating body fluid obtained from a healthy subject means that subject has or is at risk of developing AD.

In some embodiments, an amount of miR-199a-5p that is at least 1.3, at least 1.4, at least 1.5, or at least 1.6 fold higher than a base line amount of miR-199a-5p in circulating body fluid obtained from a healthy subject means that subject has or is at risk of developing AD. In some embodiments, an amount of miR-29b-3p that is at least 1.0 higher than, or at least 1.6, at least 1.8, or at least 2.0 fold lower than a base line amount of miR-29b-35p in circulating body fluid obtained from a healthy subject means that subject has or is at risk of developing AD. In some embodiments, an amount of miR-126-5p that is at least 1.0, at least 1.05, at least 1.15, or at least 1.2 fold lower than a base line amount of miR-126-5p in circulating body fluid obtained from a healthy subject means that subject has or is at risk of developing AD. In some embodiments, an amount of miR-150-5p that is at least 3.0, at least 3.3, at least 3.5, or at least 3.8 fold lower than a base line amount of miR-150-5p in circulating body fluid obtained from a healthy subject means that subject has or is at risk of developing AD.

Table 1 below provides the respective fold increases and/or decreases of additional miRNA useful in determining whether a subject has or is at risk of developing AD.

TABLE 1

| | Fold Increase/Decrease from Baseline of |
|---|---|
| | Healthy Control Indicates Subject Has |
| miRNA | or is at Risk of Developing AD |
| 151a-3p | 1.4, 1.5, 1.6, 1.7, 1.8, or 1.9 or higher |
| 151a-5p | 0.9, 1.0, 1.1, or 1.2 or higher |
| 146a-5p | 1.2, 1.3, 1.4, or 1.45 or higher |
| 4454 | −2.0, −2.1, −2.2, or −2.3 or lower |
| 10b-5p | −2.9, −3.0, −3.1, or −3.3 or lower |
| 199a-3p | 1.3, 1.4, 1.5, or 1.6 or higher |
| 199a-5p | 1.3, 1.4, 1.5, or 1.6 or higher |
| 29b-3p | 1.0 higher, or −1.6, −1.8, or −2.0 or lower |
| 126-5p | −1.0, −1.05, −1.15, or −1.2 or lower |
| 150-5p | −3.0, −3.3, −3.5, or −3.8 or lower |
| 107 | −1.2, or 1.5, 1.6, or 1.7 or higher |
| let-7g-5p | −1.0, −1.1, −1.2, or −1.3 or lower |
| 1290 | −7.0, −7.2, −7.3, or −7.4 or lower |
| 95-3p | −3.5, −3.6, −3.7, or −3.8 or lower |
| 375-3p | −1.8, −1.9, −2.0, or −2.1 or lower |
| 99a-5p | −2.2, −2.3, −2.4, or −2.5 or lower |
| 125b-5p | −2.2, −2.3, −2.4, or −2.5 or lower |
| 642a-5p | −1.0, −1.1, −1.2, or −1.3 or lower |
| 92a-1-5p | −2.3, −2.4, −2.5, or −2.6 or lower |
| 362-5p | −4.2, −4.3, −4.5, or −4.6 or lower |
| 342-3p | −2.2, −2.3, −2.4, or −2.48 or lower |
| 196a-5p | −1.7, −1.8, −1.9, or −2.0 or lower |
| 155-5p | −1.4, −1.5, −1.6, or −1.7 or lower |
| 409-3p | 2.4, 2.5, 2.6, or 2.7 or higher |
| 744-5p | 1.8, 1.9, 2.0, or 2.2 or higher |
| 223-3p | −0.8, −0.9, or −1.0, or lower |
| 584-5p | 1.2, 1.3, 1.4, or 1.5 or higher |
| 340-5p | 2.50 or higher or −3.1, −3.2, or −3.25 or lower |
| 421 | 1.1, 1.2, 1.3, or 1.4 or higher |
| 486-5p | −1.0 or lower, or 1.0 or higher |
| 126-3p | −1.4, −1.5, −1.6, or −1.7 or lower |
| 320c | 1.0 or 1.1 or higher, or −1.5, or −1.6 or lower |
| 320b | 1.1 or 1.2 or higher, or −1.5, or −1.6 or lower |
| let-7d-3p | 1.0 or 1.1 or higher, or −1.3, or −1.4 or lower |
| 27b-3p | 1.1, 1.2, 1.3, or 1.4 or higher |
| 148b-3p | 1.9 or 2.0 or higher, or −0.9, or −1.0 or lower |
| 143-3p | 0.9 or 1.0 or higher, or −20, or −21 or lower |
| 326 | 1.2, 1.3, 1.4, or 1.5 or higher |
| 335-5p | 0.7, 0.8, 0.9, or 1.0 or higher |
| 25-3p | 1.5 or 1.6 or higher, or −1.0, or −1.15 or lower |
| 153-3p | −1.0, −1.1, −1.2, or −1.25 or lower |
| 196-3p | 1.9 or 2.0 or higher, or −1.5, or −1.6 or lower |
| let-7c-5p | −0.8, −0.9, or −1.0, or lower |
| 652-3p | 1.6, 1.7, 1.8, or 1.95 or higher |
| 376c-3p | 2.1, 2.2, 2.3, 2.4, or 2.5 or higher |
| 154-5p | 1.2, 1.3, 1.4, or 1.6 or higher |
| 376a-3p | −4.7, −4.9, −5.1, or −5.2 or lower |
| 30d-5p | 1.8, 1.9, 2.0, or 2.1 or higher |
| 381-3p | 2.1, 2.2, 2.3, or 2.4 or higher |

In some embodiments, an amount of, for example miR-151a-3p that is at least 1.4, at least 1.5, at least 1.6, at least 1.7, at least 1.8, or at least 1.9 fold higher than a base line amount of miR-151a-3p in circulating body fluid obtained from a healthy subject means that the subject has or is at risk of developing MCI. In some embodiments, an amount of miR-151a-5p that is at least 0.9, at least 1.2, at least 1.3, or at least 1.7 fold higher than a base line amount of miR-151a-5p in circulating body fluid obtained from a healthy subject means that subject has or is at risk of developing MCI. In some embodiments, an amount of miR-146a-5p that is at least 1.3, at least 1.4, at least 1.5, or at least 1.6 fold higher than a base line amount of miR-146a-5p in circulating body fluid obtained from a healthy subject means that subject has or is at risk of developing MCI.

In some embodiments, an amount of miR-4454 that is at least 7.0, at least 7.1, at least 7.2, or at least 7.4 fold lower than a base line amount of miR-4454 in circulating body fluid obtained from a healthy subject means that subject has or is at risk of developing MCI. In some embodiments, an amount of miR-10b-5p that is at least 4.5, at least 4.7, at least 4.9, or at least 5.0 fold lower than a base line amount of miR-10b-5p in circulating body fluid obtained from a healthy subject means that subject has or is at risk of developing MCI. In some embodiments, an amount of miR-199a-3p that is at least 1.4, at least 1.5, at least 1.6, or at least 1.7 fold higher than a base line amount of miR-199a-3p in circulating body fluid obtained from a healthy subject means that subject has or is at risk of developing MCI.

In some embodiments, an amount of miR-199a-5p that is at least 1.7, at least 1.8, at least 2.0, or at least 2.2 fold higher than a base line amount of miR-199a-5p in circulating body fluid obtained from a healthy subject means that subject has or is at risk of developing MCI. In some embodiments, an amount of miR-29b-3p that is at least 1.0, at least 1.1, at least 1.2, or at least 1.3 fold lower than a base line amount of miR-29b-3p in circulating body fluid obtained from a healthy subject means that subject has or is at risk of developing MCI. In some embodiments, an amount of miR-126-5p that is at least 1.0, at least 1.1, at least 1.2, or at least 1.25 fold lower than a base line amount of miR-126-5p in circulating body fluid obtained from a healthy subject means that subject has or is at risk of developing MCI. In some embodiments, an amount of miR-150-5p that is at least 5.7, at least 5.8, at least 5.9, or at least 6.0 fold lower than a base line amount of miR-150-5p in circulating body fluid obtained from a healthy subject means that subject has or is at risk of developing MCI.

Table 2 below provides the respective fold increases and/or decreases of additional miRNA useful in determining whether a subject has or is at risk of developing MCI.

TABLE 2

| | Fold Increase/Decrease from Baseline of |
|---|---|
| | Healthy Control Indicates Subject Has |
| miRNA | or is at Risk of Developing MCI |
| 151a-3p | 1.4, 1.5, 1.6, 1.7, 1.8, or 1.9 or higher |
| 151a-5p | 1.8, 1.9, 2.0, 2.1, or 2.2 or higher |
| 146a-5p | 1.3, 1.4, 1.5, or 1.6 or higher |
| 4454 | −7.0, −7.1, −7.2, or −7.4 or lower |
| 10b-5p | −4.5, −4.7, −4.9, or −5.0 or lower |
| 199a-3p | 1.4, 1.5, 1.6, or 1.7 or higher |
| 199a-5p | 1.7, 1.8, 2.0, or 2.2 or higher |
| 29b-3p | −1.0, −1.1, −1.2, or −1.3 or lower |
| 126-5p | −1.0, −1.1, −1.2, or −1.25 or lower |
| 150-5p | −5.7, −5.8, −5.9, or −6.0 or lower |
| 107 | 1.4, 1.5, 1.6, or 1.7 or higher |
| let-7g-5p | 0.9 or 1.0 or higher, or −13, or −15 or lower |
| 1290 | −11, −12, −13, or −14 or lower |
| 95-3p | −3.5, −3.6, −3.7, or −3.8 or lower |
| 375-3p | −1.3, −1.4, −1.5, or −1.6 or lower |
| 99a-5p | −2.8, −2.9, −3.0, or −3.1 or lower |
| 125b-5p | −2.2, −2.3, −2.4, or −2.45 or lower |
| 642a-5p | −5.5, −5.6, −5.8, or −6.0 or lower |
| 92a-1-5p | −2.9, −3.0, −3.1, −3.2, or −3.3 or lower |
| 362-5p | −2.3, −2.4, −2.5, or −2.6 or lower |
| 342-3p | −1.8, −1.9, −2.0, or −2.1 or lower |
| 196a-5p | −1.4, −1.5, −1.6, −1.7, or −1.8 or lower |
| 155-5p | −1.0, −1.1, −1.2, or −1.3 or lower |
| 409-3p | 1.4, 1.5, 1.6, or 1.7 or higher |
| 744-5p | 1.9, 2.0, 2.1, or 2.2 or higher |
| 223-3p | 0.9, 1.2, 1.3, or 1.34 or higher |
| 584-5p | 0.9, 1.2, 1.3, or 1.4 or higher |
| 340-5p | 1.5 or 1.6 or higher, or −2.5, or −2.6 or lower |
| 421 | 0.8, 0.9, 1.0, 1.1, or 1.2 or higher |
| 486-5p | −0.7, −0.8, −0.9, or −1.0 or lower |
| 126-3p | −1.1, −1.2, −1.3, or −1.4 or lower |
| 320c | −1.8, −1.9, −2.0, −2.1, or −2.2 or lower |
| 320b | −1.7, −1.8, −1.9, or −2.0 or lower |
| let-7d-3p | −1.1, −1.2, −1.3, or −1.4 or lower |
| 27b-3p | 0.9, 1.2, 1.3, or 1.4 or higher |
| 148b-3p | 0.9, 1.2, 1.3, or 1.4 or higher |
| 143-3p | 1.5 or 1.6 or higher, or −25, or −29 or lower |
| 326 | 1.5 or 1.6 or higher, or −0.9, or −1.0 or lower |
| 335-5p | 1.1 or 1.2 or higher, or −1.1, or −1.2 or lower |
| 25-3p | 0.9 or 1.0 or higher, or −1.0, or −1.1 or lower |
| 153-3p | 1.3 or 1.4 or higher, or −1.1, or −1.2 or lower |
| 196-3p | 0.8, 0.9, 1.0, or 1.1 or higher |
| let-7c-5p | 0.7, 0.8, 0.9, or 1.0 or higher |
| 652-3p | 2.4, 2.5, 2.6, or 2.7 or higher |
| 376c-3p | 2.1, 2.2, 2.3, 2.4, or 2.5 or higher |
| 154-5p | 2.4, 2.5, 2.6, or 2.8 or higher |
| 376a-3p | 1.3, 1.4, 1.5, or 1.6 or higher |
| 30d-5p | −7.5, −7.6, −7.7, −7.8, or −7.9 or lower |
| 381-3p | 2.0, 2.1, 2.2, or 2.3 or higher |

In some embodiments, an amount of, for example miR-151a-3p that is at least-0.7, at least-0.8, at least-0.9, at least-1.0 fold lower than a base line amount of miR-151a-3p in circulating body fluid obtained from a subject previously diagnosed with MCI means that the subject has or is at risk of progressing from MCI to AD. In some embodiments, an amount of miR-151a-5p that is at least-1.4, at least-1.5, at least-1.6, at least-1.7, or at least-1.8 fold lower than a base line amount of miR-151a-5p in circulating body fluid obtained from a subject previously diagnosed with MCI means that the subject has or is at risk of progressing from MCI to AD. In some embodiments, an amount of miR-146a-5p that is at least 1.2, or at least 1.3 fold higher, or at least-1.0 or at least-1.1 fold lower than a base line amount of miR-146a-5p in circulating body fluid obtained from a subject previously diagnosed with MCI means that the subject has or is at risk of progressing from MCI to AD.

Table 3 below provides the respective fold increases and/or decreases of additional miRNA useful in determining whether a subject previously diagnosed with MCI has or is at risk of progressing from MCI to AD or at risk of increasing AD severity.

TABLE 3

| | Fold Increase/Decrease from MCI Subjects Indicates |
|---|---|
| | Subject Has or is at Risk of Progressing from MCI |
| miRNA | to AD or at risk of increasing AD severity |
| 151a-3p | −0.7, −0.8, −0.9, or −1.0 or lower |
| 151a-5p | −1.4, −1.5, −1.6, −1.7, or −1.8 or lower |
| 146a-5p | 1.2 or 1.3 or higher, or −1.0, or −1.1 or lower |
| 4454 | 1.7, 1.8, 1.9, or 2.1 or higher |
| 10b-5p | 0.9, 1.1, 1.2, or 1.3 or higher |
| 199a-3p | −0.7, −0.8, −0.9, or −1.0 or lower |
| 199a-5p | −1.0, −1.1, −1.2, or −1.3 or lower |
| 29b-3p | 1.2 or 1.3 or higher, or −1.4, or −1.5 or lower |
| 126-5p | 0.7, 0.8, 0.9, or 1.0 or higher |
| 150-5p | 0.7, 0.8, 0.9, or 1.0 or higher |
| 107 | −1.7, −1.8, −1.9, or −2.0 or lower |
| let-7g-5p | 1.0 or 1.1 or higher, or −1.2, or −1.3 or lower |
| 1290 | 1.6, 1.7, 1.8, 1.9, or 2.0 or higher |
| 95-3p | 1.9 or 2.0 or higher, or −0.9, or −1.0 or lower |
| 375-3p | 1.5 or 1.6 or higher, or −1.1, or −1.2 or lower |
| 99a-5p | 0.8, 0.9, 1.0, or 1.2 or higher |
| 125b-5p | 1.4 or 1.5 or higher, or −0.9, or −1.0 or lower |
| 642a-5p | 0.8, 0.9, 1.0, 1.2, or 1.3 or higher |
| 92a-1-5p | 1.1 or 1.2 or higher, or −0.9, or −1.0 or lower |
| 362-5p | 2.0 or 2.1 or higher, or −1.6, or −1.7 or lower |
| 342-3p | 1.0 or 1.1 or higher, or −1.0, or −1.1 or lower |
| 196a-5p | 1.1 or 1.2 or higher, or −1.4, or −1.5 or lower |
| 155-5p | −0.8, −0.9, −1.1, or −1.2 or lower |
| 409-3p | 1.7 or 1.8 or higher, or −1.4, or −1.5 or lower |
| 744-5p | 0.9 or 1.0 or higher, or −1.6, or −1.7 or lower |
| 223-3p | −0.7, −0.8, −0.9, or −1.1 or lower |
| 584-5p | 0.7, 0.8, 0.9, or 1.0 or higher |
| 340-5p | 1.4 or 1.5 or higher, or −1.1, or −1.2 or lower |
| 421 | 0.8, 0.9, 1.0, or 1.1 or higher |
| 486-5p | 0.7, 0.8, 0.9, or 1.0 or higher |
| 126-3p | −0.8, −0.9, −1.1, or −1.2 or lower |
| 320c | 1.3, 1.4, 1.5, or 1.6 or higher |
| 320b | 1.1, 1.2, 1.3, or 1.4 or higher |
| let-7d-3p | 0.8, 0.9, 1.0, or 1.1 or higher |
| 27b-3p | −0.6, −0.7, −0.8, or −1.0 or lower |
| 148b-3p | 1.2 or 1.3 or higher, or −1.5, or −1.6 or lower |
| 143-3p | 1.2 or 1.3 or higher, or −1.4, or −1.5 or lower |
| 326 | 2.0 or 2.1 or higher, or −0.9, or −1.0 or lower |
| 335-5p | 0.8, 0.9, 1.0, or 1.2 or higher |
| 25-3p | 1.8 or 1.9 or higher, or −1.1, or −1.2 or lower |
| 153-3p | −0.8, −0.9, −1.0, or −1.1 or lower |
| 196-3p | 1.4 or 1.5 or higher, or −1.8, or −1.9 or lower |
| let-7c-5p | −0.8, −0.9, −1.1, or −1.2 or lower |
| 652-3p | −1.1, −1.2, −1.3, or −1.4 or lower |
| 376c-3p | −0.7, −0.8, −0.9, or −1.1 or lower |
| 154-5p | −0.6, −0.7, −0.8, or −1.0 or lower |
| 376a-3p | 1.2, 1.3, 1.4, or 1.5 or higher |
| 30d-5p | −0.7, −0.8, −0.9, or −1.1 or lower |
| 381-3p | −1.3, −1.4, −1.5, or −1.6 or lower |

The term “baseline amount” as used herein refers to an average, mean, or absolute amount of one or more miRNAs obtained from circulating bodily fluid obtained from one or more suitable control subjects. For example, a control subject can be a subject that does not have a cognitive disorder. In certain embodiments, a control subject is a subject who does not have AD and/or MCI. Typically, such healthy subjects are young adults (e.g., within the ages of 18-30) that show no signs or symptoms of a cognitive disorder and/or have no family history of a cognitive disorder.

In some embodiments, a comparison determines that the amount of one or more miRNAs present in circulating bodily fluid obtained from a first subject is from about 1.0-fold to about 25-fold higher or lower than the amount of such one or more miRNAs present in circulating bodily fluid obtained from a control subject, (the control subject may be a subject previously diagnosed with MCI, when using the methods disclosed herein to assess whether that subject has or is at risk of progressing from MCI to AD), thereby indicating that the first subject has AD and/or MCI or has a statistical likelihood of developing AD and/or MCI. In some embodiments, such a comparison determines that the amount of one or more of the miRNAs: 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p present in circulating bodily fluid obtained from a first subject is from about 1.0-fold to about 25-fold higher or lower than the amount of such one or more miRNAs present in circulating bodily fluid obtained from a control subject, thereby indicating that the first subject has AD and/or MCI or has a statistical likelihood of developing AD and/or MCI.

In some embodiments, the presence or absence of AD and/or MCI in a subject is determined according to an amount of one or more miRNAs that is associated with circulating bodily fluid obtained from a subject. In some embodiments, an amount of at least 0.01 mean normalized copy number/μl of miRNA, at least 0.05 mean normalized copy number/μl of miRNA, at least 0.1 mean normalized copy number/μl of miRNA, at least 0.5 mean normalized copy number/μl of miRNA, at least 1 mean normalized copy number/μl of miRNA, at least 3 mean normalized copy number/μl of miRNA, at least 5 mean normalized copy number/μl of miRNA, at least 10 mean normalized copy number/μl of miRNA, or more of miRNA that is associated with circulating bodily fluid obtained from a subject indicates that the subject has AD and/or MCI or has a statistical likelihood (i.e., is “at risk”) of developing AD and/or MCI. In other embodiments, an amount of at least 50 μg/μL of miRNA, at least 100 μg/μL of miRNA, at least 150 μg/μL of miRNA, at least 200 μg/μL of miRNA, at least 250 μg/μL of miRNA, at least 300 μg/μL of miRNA, at least 350 μg/μL of miRNA, at least 400 μg/μL of miRNA, at least 450 pg/μL of miRNA, at least 500 μg/μL of miRNA, at least 550 μg/μL of miRNA, at least 600 pg/μL of miRNA, at least 650 μg/μL of miRNA, at least 700 μg/μL of miRNA, at least 750 pg/μL of miRNA, at least 800 μg/μL of miRNA, at least 850 μg/μL of miRNA, at least 900 pg/μL of miRNA, at least 950 μg/μL of miRNA, at least 1000 μg/μL of miRNA, or more of miRNA that is associated with circulating bodily fluid obtained from a subject indicates that the subject has AD and/or MCI or has a statistical likelihood (i.e., is “at risk”) of developing AD and/or MCI. In an embodiment, the one or more miRNAs are selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p.

In some embodiments, methods are provided for monitoring the progression of AD and/or MCI in a subject. In some embodiments, such a method comprises a) obtaining a circulating bodily fluid sample from the subject; (b) determining an amount of one or more miRNAs present in the circulating bodily fluid, such as one or more miRNAs selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p, without determining an amount from a subject's neural-derived exosomes; and (c) comparing the amount of the one or more miRNAs determined in step (b) to a baseline amount of the one or more miRNAs; and (d) determining the progression or lack of progression of AD and/or MCI based on the comparison in (c).

In some embodiments, methods are provided for monitoring a response to treatment of AD and/or MCI in a subject. In some embodiments, such a method comprises a) obtaining a circulating bodily fluid sample from the subject; (b) determining an amount of one or more miRNAs present in the circulating bodily fluid, such as one or more miRNAs selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p without determining an amount from a subject's neural-derived exosomes; (c) comparing the amount of the one or more miRNAs determined in step (b) to a baseline amount of the one or more miRNAs; and (d) monitoring the response to treatment based on the comparison in (c). In some embodiments, a difference between an amount of one or more miRNAs from the circulating bodily fluid obtained from a subject after treatment of the subject has commenced and a baseline amount that is less than the difference obtained at an earlier point in time indicates that the subject has responded favorably to the treatment. In some embodiments, a difference between an amount of one or more miRNAs from the circulating bodily fluid obtained from a subject and a baseline amount that is greater than the difference obtained at an earlier point in time indicates that the subject has not responded favorably to the treatment.

In some embodiments, the baseline amount may be an amount considered ‘normal’ for the particular miRNA (e.g., an average amount for age-matched individuals not diagnosed with the cognitive disorder), or the baseline amount may be a historical reference amount for the particular subject (e.g., a baseline amount that was obtained from a circulating bodily fluid sample derived from the same subject, but at an earlier point in time). Quantitative baseline amounts that are determined contemporaneously (e.g., a reference value that is derived from a pool of samples including the sample being tested) are also contemplated. Accordingly, in some embodiments, methods are provided for monitoring progression of AD and/or MCI in a subject by obtaining a quantitative measured amount for one or more miRNAs present in circulating bodily fluid obtained from a sample and comparing such measured value to a baseline amount. In some embodiments, a difference between an amount of one or more miRNAs from circulating bodily fluid obtained from a subject and a baseline amount that is less than the difference obtained at an earlier point in time indicates that the progression of the disease has diminished. In some embodiments, a difference between an amount of one or more miRNAs from circulating bodily fluid obtained from a subject and a baseline amount that is greater than the difference obtained at an earlier point in time indicates that the progression of the disease has increased.

### Administration

Any suitable method of administering a treatment or drug to a subject can be used. Any suitable formulation and/or route of administration can be used for administration of a treatment or drug disclosed herein (e.g., see Fingl et al. 1975, in “The Pharmacological Basis of Therapeutics”, which is incorporated herein by reference in its entirety). A suitable formulation and/or route of administration can be chosen by a medical professional (e.g., a physician) in view of, for example, a subject's disease, condition, symptoms, weight, age, and/or general health. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments administering a drug to a subject comprises providing the drug to the subject, for example for self-administration or for administration to the subject by another (e.g., by a non-medical professional). As another example, a drug can be provided as an instruction written by a medical practitioner that authorizes a patient to be provided a drug or treatment described herein (e.g., a prescription). In yet another example, a drug can be provided to a subject where the subject self-administers a composition orally, intravenously or by way of an inhaler, for example. Alternately, one can administer a drug in a local rather than systemic manner, for example, via direct application to the skin, mucous membrane or region of interest for treating, including using a depot or sustained release formulation. In certain embodiments a drug is administered alone (e.g., as a single active ingredient (AI) or, e.g., as a single active pharmaceutical ingredient (API)). In other embodiments, a drug is administered in combination with one or more additional AIs/APIs, for example, as two separate compositions or as a single composition where the one or more additional AIs/APIs are mixed or formulated together with a drug in a pharmaceutical composition.

In some embodiments, an amount of AD and/or MCI drug administered to a subject is a therapeutically effective amount. In some embodiments, a therapeutically effective amount of a drug is an amount needed to obtain an effective therapeutic outcome. In certain embodiments, a therapeutically effective amount of a drug is an amount sufficient to treat, reduce the severity of, inhibit or delay the onset of, mitigate and/or alleviate one or more symptoms of AD and/or MCI. Determination of a therapeutically effective amount is well within the capability of those skilled in the art, especially in light of the detailed disclosure provided herein.

In certain embodiments, a therapeutically effective amount is an amount high enough to provide an effective therapeutic effect (e.g., a beneficial therapeutic effect) and an amount low enough to minimize unwanted adverse reactions. Accordingly, in certain embodiments, a therapeutically effective amount of a drug may vary from subject to subject, often depending on age, weight, general health condition of a subject, severity of a condition being treated and/or a particular combination of drugs administered to a subject. Thus, in some embodiments, a therapeutically effective amount is determined empirically. Accordingly, in certain embodiments, a therapeutically effective amount of a drug that is administered to a subject can be determined by one of ordinary skill in the art based on amounts found effective in animal or clinical studies, a physician's experience, and/or suggested dose ranges or dosing guidelines.

In certain embodiments, a therapeutically effective amount of L-serine or a composition disclosed herein comprises one or more doses (administered to a subject) comprising at least 0.1 mg/kg, at least 5 mg/kg, at least 10 mg/kg, at least 15 mg/kg, at least 20 mg/kg, at least 25 mg/kg, at least 50 mg/kg, at least 100 mg/kg, at least 250 mg/kg, at least 500 mg/kg, at least 1000 mg/kg, at least 5000 mg/kg, or at least 7500 mg/kg of L-serine, or a salt, a precursor, derivative or conjugate thereof, per kg body weight of a subject.

In some embodiments administering a therapeutically effective amount of an AD and/or MCI drug or composition disclosed herein comprises administering a suitable dose hourly, every two hours, every 4 hours, every 6 hours, every 8 hours, or every 12 hours. In certain embodiments, a cognitive disorder drug can be administered at least one, at least two, at least three, at least four, at least five, or at least six times per day, e.g., 1 to 12 times per day, 1 to 8 times per day, or 1 to 4 times per day per day. In certain embodiments, a cognitive disorder drug disclosed herein can be administered once, twice, 3 times, 4 times, 5 times, 6 times, 7 times, 8 times, 9 times, 10 times, 11 times, or 12 times per day. A cognitive disorder drug may be administered in a single dosage form or one or more dosage forms. A daily dose can be achieved in the form of a single dose or in the form of a plurality of partial doses.

An AD and/or MCI drug may be administered on a daily basis or on a schedule containing days where dosing does not take place. For example, dosing may take place every other day, or dosing may take place for 2, 3, 4, or 5 consecutive days of a week, then be followed by from 1 to 5 non-dosing days.

An AD and/or MCI drug can be administered for at least a day, at least two days, at least three days, at least four days, at least five days, at least a week, at least two weeks, at least three weeks, at least a month, at least two months, at least three months, at least six months, at least a year, at least two years, or more, or for any extended duration to further improve, maintain, or retain therapeutic efficacy. In certain embodiments, a cognitive disorder drug is administered for a duration of 1 week to 10 years or more. In some embodiments administering a therapeutically effective amount of a drug, or a pharmaceutical composition comprising a drug, comprises administering a suitable dose at a frequency or interval as needed to obtain an effective therapeutic outcome. In some embodiments administering a therapeutically effective amount of a drug or a pharmaceutical composition disclosed herein comprises administering a suitable dose hourly, every two hours, every 4 hours, every 6 hours, three times a day, twice a day, once a day, six times a week, five times a week, four times a week, three times a week, twice a week, weekly, at combinations thereof, and/or at regular or irregular intervals thereof, and/or simply at a frequency or interval as needed or recommended by a medical professional. In some embodiments, a therapeutically effective amount of a drug or a pharmaceutical composition comprising a therapeutically effective amount of drug is administered continuously by, for example by intravenous administration.

### Kits

In some aspects, presented herein is one or more kits useful in carrying out a method of identifying a subject who has, or is at risk of developing AD and/or MCI, or identifying subjects that are or are at risk of progressing from MCI to AD, in which the kit or kits includes one or more multi-well plates, with each well contains one or more miRNA primers selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p, and a mechanism to calculate the likelihood of a positive or negative diagnosis, and/or probability of having or not having a neurological disease. In one embodiment, the kit includes two well-plates in which one well plate includes each well containing one or more miRNA primers selected from the list above, and the other well plate includes a Quality Control (QC)/spike-in/calibration plate in which one or more wells contain QC primers, no-template controls, and/or spike-in primers designed to monitor the success of the method. In another embodiment, the kit includes one well-plate in which the wells include one or more miRNA primers selected from the list above, QC primers, no-template controls, and/or spike-in primers designed to monitor the success of the method. In another embodiment, the kit includes one well-plate in which the wells include one or more miRNA primers selected from the list above. In a further embodiment, the mechanism to calculate the likelihood of a positive or negative diagnosis, and/or probability of having or not having a neurological disease or disorder includes software configured to carry out the calculation using random forest machine learning algorithms and/or logistic regression algorithms developed from machine learning classification of known disease and healthy control plasma samples.

The calculation may be performed using random forest machine learning algorithms and/or logistic regression algorithms developed from machine learning classification of known disease and healthy control plasma samples. Random forest machine learning algorithms are well-known and described in the art, and typically rely on random forest algorithms that have three main hyperparameters: (i) node size; (ii) number of trees; and (iii) number of features sampled, that are predetermined. Logistic regression machine learning algorithms also are well known in the art and are used to accomplish binary classification tasks by predicting the probability of an outcome, event, or observation. Using the techniques described herein, those skilled in the art will be capable of using random forest machine learning algorithms and/or logistic regression algorithms to develop software or a protocol for carrying out the method of identifying a subject who has, or is at risk of developing a motor neuron disease and/or neurodegenerative disorder (specifically AD and/or MCI) using the presence or absence of two or more, three or more, four or more, five or more, six or more, seven or more, or up to all of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p. The software can be provided with one or more kit(s) described herein the first time the purchaser acquires a kit, and loaded onto a computing device integral to, incorporated into, or in communication with a device for conducting qPCR

The well-plates useful in the kits described herein may include, for example, qPCR plates of varying sizes, including 48, 96, and 384-well plates. Each of the respective wells can pre-loaded with one or more of the miRNA primers selected from 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p, and/or QC primers, no-template controls, and/or spike-ins. Particularly preferred QC primers, no-template controls, and/or spike-in primers that may be used in the kit(s) described herein are described in more detail in the examples below. In one embodiment, a kit may contain one or two 96-well plates suitable for 10 μL or 25 μL reactions in which one or two of the well-plates contains wells filled with one or more of the miRNA primers selected from 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and QC primers, no-template controls, and/or spike-in primers. In another embodiment, a kit may contain one 96-well plates suitable for 10 μL or 25 μL reactions in which the well-plates are filled with one or more of the miRNA primers detailed above. In another embodiment, the kit includes a 384-well plate suitable for 10 μL reactions, in which all of the miRNA, QC primers, no-template controls, and/or spike-in primers are included on the same plate or one 384-well plate with only primers from above, or two plates in which all of the miRNA, QC primers, no-template controls, and/or spike-in primers are included across the two plates.

The kits are particularly suited for automated pipetting using a liquid handling robot (e.g., an Opentrons OT-2 robot) for sample loading into the respective wells in the plate(s) of the respective kit(s) that are preloaded with the reagents discussed above. Operators supply the RNA/cDNA. The kits can be designed for use in any analyzer capable of carrying out qPCR analysis on the circulating bodily fluid samples added to the respective well-plates, and indicating a presence and/or amount of one or more of the miRNA primers selected from 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p. The method of diagnosis can be carried out using the information generated regarding the presence and/or amount of one ore more of the miRNAs selected from 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p.

The diagnostic method preferably is carried out on-site using a mechanism for calculating the likelihood of a positive or negative diagnosis, and/or probability of having or not having a neurological disease. A preferred device for conducting the qPCR includes the use of a convention qPCR real time diagnostic system, such as (but not limited to) those available as BioRad CFX series, Applied Biosystems, Edvotek, MSE PRO series, Benchmark Scientific, and the like. The data from the qPCR system can be fed to a computing apparatus configured to calculate the likelihood of a positive or negative diagnosis, and/or probability of having or not having a neurological disease.

The calculation may be performed using random forest machine learning algorithms, logistic regression algorithms (eg., cross-validation models, Lasso, glm, glm-net, etc.), and/or other algorithms developed from machine learning classification of known disease and healthy control plasma samples, or plasma from other disease classes. Using the guidelines provided herein, those skilled in the art are capable of using random forest machine learning algorithms and/or logistic regression algorithms to develop software or a protocol for carrying out the method of identifying a subject who has, or is at risk of developing a cognitive disorder (AD and/or MCI) using the presence or absence of two more of miRNA selected from 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p. The software can be provided with one or more kit(s) described herein the first time the purchaser acquires a kit, and loaded onto a computing device integral to, incorporated into, or in communication with a device for conducting qPCR.

### The Model

Certain embodiments also relate to a model and system useful in detecting the presence or absence of AD and/or MCI. The model can be developed using samples obtained from healthy individuals and from individuals known to have AD and/or MCI in which the samples are processed to extract RNA, synthesize cDNA, amplify the RNA using, for example, qPCR, and assessing the differences in expression of various miRNA between healthy individuals and those known to have AD and/or MCI. The model can be created by comparing miRNA from AD and/or MCI subjects to healthy control subjects, assessing the fold differences between each miRNA from AD and/or MCI subjects and healthy control subjects, and generating a list of miRNA that exhibited sufficiently consistent fold changes between AD and/or MCI subjects and healthy controls. Sufficiently consistent fold changes denote a fold change of at least 1.0 between either AD or MCI and healthy controls, as well as whether the miRNA were upregulated or downregulated. For example, if miRNA 151a-3p was consistently upregulated with a fold change of more than 1.5 in AD subjects when compared to healthy controls, then miRNA 151a-3p would be included in the AD list. Similarly, if miRNA 4454 was consistently downregulated by more than 2.0 in AD subjects when compared to healthy controls, then miRNA 4454 would be included in the AD list.

Using the miRNA identified above as having sufficiently consistent fold changes, the model can be further developed by quantifying the abundance of each of the miRNAs identified in the model using qPCR techniques. The raw cycle quantitation (Cq) numbers can then be normalized by the geomean of three endogenous miRNA, thus generating a deltaCq value for each miRNA. These deltaCq values may then be used in a logistic regression analysis, using a Lasso selection feature to determine which of the miRNA from the list above contributed the most to separating AD, MCI, and healthy controls, and then assigning a coefficient to each of the selected miRNA based on the relative contribution of each miRNA in separating AD, MCI, and healthy control subjects. The logistic regression analysis may be carried out by conducting a relaxed fit, no penalty, multinomial logistic regression (glmnet) in R-studio with four libraries: data.table, glmnet, knitr, and caret. The regression used a lasso feature selection which kept out-of-fold predictions and included the following parameters: seed set of 42, multinomial family, grouped type, mse measure, Z-score standardization, intercept included, lambda sequence (exp(seq(−4,−1, length.out=1000), 20 nfolds, convergence threshold of 1e-6, 1e7 maximum iterations. The analysis creates a select list of miRNA that contributed the most to distinguishing between disease and healthy subject, and assigned a coefficient to each miRNA.

Using this model, the combined qPCR data from a single individual can then be compared to the model to determine a disease classification prediction for that individual. Specifically, the raw cycle quantitation (Cq) numbers for the list of miRNA developed in the model can then be normalized by the geomean of three endogenous miRNA, thus generating a deltaCq value for each miRNA, using the techniques discussed in the examples hereinabove. These deltaCq values are then compared to the logistic regression model developed from the database to determine the prediction of disease state incrementally using the value of each miRNA. In the multinomial model, a positive coefficient means that a one-unit increase in the value for that specific miRNA increases the log-odds of the individual having disease A, (e.g., AD), and decreases the log-odds of the person having disease B (e.g, MCI) and C (healthy), relative to the overall average. When the coefficient is negative, a one-unit increase in the value for that specific miRNA decreases the log-odds of the individual having disease A. The combination of log-odds for each miRNA in the model (e.g., the sum of the log-odds calculated based on the coefficients) determines the final prediction that a person does or does not have the disease. The model adds up the likelihood that the person has A (e.g., AD) by adding up each of the log odds ratios calculated from the coefficients assigned to each miRNA in the model. It then generates a prediction number that indicates that the person is predicted to have the disease with a certain percentage certainty, (e.g., person has AD with 95% certainty).

The model of the embodiments therefore includes at least: (1) a database stored in a storage medium, wherein the database comprises dCq values for individuals with a known disease status, a list of miRNA and a coefficient associated with each miRNA, wherein the list of miRNA is selected from 4 or more miRNA selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p, and the coefficient is used to calculate the log-odds that the subject has a given disease state (or does not have a certain disease state); and (2) software configured to access the database, process qPCR generated quantification data for each miRNA in the list; generate normalized qPCR values for each miRNA in the list, and process the normalized qPCR values for each miRNA by computing and outputting a predicted diagnosis of AD and/or MCI using the software. The predictive certainty uses parameters pre-set into the software to provide the computation.

In one embodiment, the database of the model includes a list of miRNA useful in distinguishing between subjects having AD, subjects having MCI, and healthy subjects, in which the list of miRNA is selected from 4 or more miRNA selected from the group consisting of 151a-3p, 199a-5p, 150-5p, 375-3p, 99a-5p, 642a-5p, 92a-1-5p, 223-3p, 421, 320b, 335-5p, 153-3p, 19b-3p, 652-3p, and 30d-5p. In an embodiment, the list of miRNA is selected from 5 or more, or 6 or more, or 7 or more, or 8 or more, or 9 or more, or 10 or more, or 11 or more, or 12 or more, or 13 or more, or 14 or more, or 15 or more, or all 16 of the miRNA selected from the above list.

In another embodiment, the database of the model includes a list of miRNA useful in distinguishing between subjects having AD and subjects having MCI, in which the list of miRNA is selected from 4 or more miRNA selected from the group consisting of 4454, 95-3p, 375-3p, 642a-5p, 92a-1-5p, 196a-5p, 223-3p, 421, 320b, 335-5p, and 30d-5p. In an embodiment, the list of miRNA is selected from 5 or more, or 6 or more, or 7 or more, or 8 or more, or 9 or more, or 10 or more, or all 11 miRNA from the above list.

Another embodiment includes a method of creating a model suitable for use in diagnosing the presence or absence of AD and/or MCI. The method includes a method of generating a database comprising a set of miRNA and coefficients corresponding to each miRNA in the set, and creating software configured to process miRNA data from a sample from a subject and generate a diagnosis of whether the subject has, is susceptible to having, or does not have AD and/or MCI. The method of generating the database may include obtaining samples from subjects known to have AD, known to have MCI, and known to be healthy, extracting miRNA from exosomes from the respective samples, synthesizing cDNA for each miRNA, amplifying the miRNA using, for example, qPCR to determine the quantification cycle (Cq) values for each miRNA. The database may be further created by normalizing the Cq values for each of miRNA using the geometric mean of three stable reference miRNA, and determining the gene-fold expression of each miRNA from the delta Cq values.

The database can further be created by assessing the differences in expression of the extracted miRNA between healthy individuals and those known to have AD and/or MCI. The database can be created by comparing miRNA from AD and/or MCI subjects to healthy control subjects, assessing the fold differences between each miRNA from AD and/or MCI subjects and healthy control subjects, and generating an initial list of miRNA that exhibited sufficiently consistent fold changes between AD and/or MCI subjects and healthy controls. Sufficiently consistent fold changes denote a fold change of at least 1.0 between either AD or MCI and healthy controls, as well as whether the miRNA were upregulated or downregulated.

The database can be further created by conducting a logistic regression analysis on the initial list of miRNA by subjecting the delta Cq values for each of the miRNA in the initial list to an iterative regression analysis using a Lasso selection feature by comparing delta Cq values of miRNA from known AD and/or MCI subject to delta Cq values of miRNA from known healthy controls. The iterative regression analysis with the Lasso selection feature can determine which miRNA contribute the most to AD and/or MCI, and which miRNA contribute the most to healthy controls, and will generate a second, smaller list of miRNA from the above initial list, along with a coefficient associated with each miRNA in the second list that represents the relative contribution of each miRNA in separating AD, MCI, and healthy control subjects.

The iterative regression analysis with the Lasso selection feature ends when the predictive accuracy of AD, MCI, or healthy is at or above about 80%, thereby generating a database that includes a second list of miRNA and coefficients corresponding to each miRNA. The completion of the regression analysis also establishes the parameters in the software for carrying out subsequent regression analyses on samples from unknown subjects, thereby creating the software configured to process miRNA data from a sample from a subject and generate a diagnosis of whether the subject has, is susceptible to having, or does not have AD and/or MCI. The model can be continuously updated by repeating the above method with additional samples from subjects known to have AD, MCI, or from healthy controls, where the updated model may create minor modifications to the miRNA in the second list along with their respective coefficients, and modify the parameters in the software.

The embodiments also include a method of using the model to diagnose a subject as either having or being susceptible of developing AD and/or MCI, or as not having or being susceptible of developing AD and/or MCI. The method includes obtaining a sample from a subject, extracting miRNA from the sample, synthesizing cDNA for each miRNA listed in the model (e.g., those miRNA from the second list), amplifying the miRNA using, for example, qPCR and determining the quantification cycle (Cq) values for each miRNA. The raw cycle quantitation (Cq) numbers for the miRNA developed in the model can then be normalized by the geomean of three endogenous miRNA, thus generating a deltaCq value for each miRNA, using the techniques discussed above. These deltaCq values are then input into the software created in developing the model in which they are subjected to a mathematical comparison to the logistic regression analysis with its predetermined parameters generated in developing the model.

The system may then determine how each miRNA value contributes to the prediction of disease state incrementally and will generate a coefficient for each miRNA that is indicative of the log-odds of the subject having or being susceptible of developing AD and/or MCI, or as not having or being susceptible of developing AD and/or MCI. The method may further include combining the log-odds for each miRNA in the model to generate the final prediction that a person does or does not have, or is not susceptible to having AD and/or MCI, in accordance with the following equation 1 below, a linear regression model used in predictive modeling. For example, the software may add up each of the log odds ratios using the coefficients assigned to each miRNA in the model, and then generate a prediction number that indicates that the person is predicted to have the disease with a certain percentage certainty, (e.g., person has AD). with 95% certainty).

$$\begin{matrix}{p}_{j} ( x ) := \text{?} [ Y = j \vert {X}_{1} = {x}_{1} , … , {X}_{p} = {x}_{p} ] \\ = \frac{{e}^{{β}_{0 j} + {β}_{1 j} {X}_{1} + ⋯ + {β}_{pj} {X}_{p}}}{1 + {\sum }_{ℓ = 1}^{J = 1} {e}^{{β}_{0 ℓ} + {β}_{1 ℓ} {X}_{1} + ⋯ + {β}_{p ℓ} {X}_{p}}}\end{matrix} \qquad [ Equation 1 ]$$

In Equation 1, pj(x) is the prediction of disease state, Y represents categorical data variables, {1, . . . , J} represents the disease groups, and X1, . . . . Xp represents the miRNA predictors. Information regarding predictive modeling is available, for example, at egarpor.github.io/PM-UC3M, incorporated herein by reference in its entirety.

### EXAMPLES

The Examples of U.S. Patent Application Publication No. 2021/0164051 are incorporated herein by reference in their entirety. The Examples of our earlier application provide detailed information regarding the use of neural derived exosomes to determine the presence or amount of one or more, or combinations of certain miRNA associated with the neural derived exosomes. These Examples provide additional information regarding the determination of whether a distinct difference in the presence and/or amounts of one or more, or combinations of certain miRNA associated with the neural derived exosomes is found between healthy, or control, subjects, and subjects known to have a cognitive disorder.

### Example 1—Developing an miRNA Fingerprint of miRNA for Alzheimer's Disease and/or Mild Cognitive Impairment

Plasma samples were collected in K2EDTA tubes from a total of 126 AD subjects, 66 MCI subjects, and 224 healthy subjects and were separated into three cohort groups. The first Cohort included 39 AD, 39 MCI and 42 control samples; the second Cohort included 62 AD, 21 MCI and 82 control samples; and the third Cohort included 25 AD, 6 MCI, and 100 control samples. The AD samples were obtained from Precision for Medicine (Norton, MA, CR00425931, collected across the USA) and Dartmouth Hitchcock Medical School (NCT03062449), MCI samples were obtained from Houston Methodist's Neurological Institute (HMCI-2), and healthy control samples were obtained from Innovative Research Inc (Novi, MI, FDA Approval, #3003372368, collected from across the USA) and Precision for Medicine (Norton, MA, CR00425931, collected from across the USA). All blood samples were collected in K2EDTA tubes and samples blinded before being processed and analyzed. Every patient sample was from a unique individual with no overlap between cohorts.

### RNA Extraction

Control and AD and/or MCI plasma was removed from an-80 °C freezer, thawed at room temperature, then 200 μL aliquoted into a 1.5 mL LoBind® Tube (Eppendorf). Total RNA was extracted using the Qiagen miRNeasy Serum/Plasma Kit (50) Cat. No./ID: 217184 according to the manufacturer's instructions. Briefly, 1 mL Qiazol Lysis buffer, containing 1 μL UniSp2, 4 and 5 spike-in, was added to each sample and mixed by pipetting up and down several times. Samples were incubated at room temperature (RT) for 5 mins. An equal volume of chloroform (200 μL) was added to each sample, then the tubes were vigorously shaken for 15 seconds. The samples were incubated at RT for 3 mins, then centrifuged at 12,000×g for 15 min at 4 °C, to generate three distinct layers. The top aqueous layer containing RNA was carefully removed without touching the middle layer containing DNA and placed in a new clean 2 mL LoBind® tube. To this were added 1.5 volumes of 100% ethanol (to 700 μL RNA was added 1050 μL 100% ethanol). The samples were mixed thoroughly by pipetting up and down. To a spin column, 700 μL sample was added, then centrifuged at 10,000×g for 15 secs at RT, then the eluent was discarded. This step was repeated until all the sample had been added to the spin column (three spins).

About 700 μL Buffer RWT then was pipetted into each column, then spun at 10,000×g for 15 secs at RT. The eluent was discarded. Next, about 500 μL of Buffer RPE was added to the spin-column then centrifuged at 10,000×g for 15 secs at RT, and the eluent was discarded. About 500 μL 80% ethanol then was added to the spin column then spun for 2 min at 10,000×g at RT, and the eluent discarded. The spin-column was placed in a new 2 mL collection tube (with no lid), and centrifuged at 17,000×g for 5 min, RT to dry the spin column. Finally, the spin column was placed in a clean 1.5 mL LoBind® tube, and 15 μL nuclease-free water added directly to the membrane. The spin-columns were incubated at RT for 1 min, then spun for 1 min at 17,000×g to elute the RNA. The RNA was either immediately used to make cDNA, or frozen at −80 °C until required.

### CDNA Synthesis

cDNA was synthesised as described previously (Dunlop et al 2021), and according to the manufacturer's instructions using the Qiagen miRCURY LNA RT Kit (#339340). cDNA was synthesised in duplicate, and to each 10 μL reaction were added 0.5 μL of UniSp6 and cel-miR-39-3p spike-ins (UniSp6 is a part of the miRCURY LNA RT Kit #339340 and cel-miR-39-3p is part of the miRCURY LNA RNA Spike-in Kit #339390. For instructions on how to reconstitute these, see RNA Spike-in Kit, for RT, Handbook #HB-2433-002). About 4 μL total RNA was used in the cDNA synthesis reaction, since it was previously demonstrated that 4 μL returns a more robust Cq in genes of low copy number (Dunlop et al., 2021). The conditions for the RT reaction were as follows: 95 °C 5 min, 42 °C 60 mins, 4 °C HOLD. Duplicate cDNA reactions were pooled into a final volume of 20 μl, then aliquoted into 3 μL aliquots before being stored at −20 °C.

For each sample, quality control was conducted prior to downstream qPCR analysis. Each sample was assayed for the following spike-ins; UniSp2, 4 and 5 to measure the effectiveness of the RNA extraction step, and UniSp6 and cel-miR-39-3p to measure the success of the cDNA/RT reaction step. All samples were deemed to have passed QC if the Cqs for spike-ins varied by less than two standard deviations from the mean raw Cq for each target.

### Sample Signal

As a method for determining whether there was sufficient cDNA in each sample to proceed with downstream reactions, four miRNAs were measured that were collectively referred to as “sample signal.” Four miRNA that were predicted to be either in low or high copy-number in human plasma were measured; miR-142-3p, miR-451a, miR-23a-3p and miR-103a-3p. The presence of these targets with raw Cqs less than 34 constituted a “pass” for use in downstream experiments.

### Hemolysis Calculation

Certain miRNAs are enriched in red blood cells, and if hemolysis occurs during plasma processing, the release of these miRNAs into the plasma can artefactually affect results. Thus, miR-451a, miR-23a-3p were measured, and delta Cq for miR-23a-3p-miR-451a was calculated. Any delta Cq that was >7 indicated hemolysis may have occurred and these samples would be assessed.

Real-Time PCR (qPCR) and Analysis

cDNA was removed from the freezer (−20 °C) and diluted 1/30 into nuclease-free water. qPCR was conducted using Qiagen miRCURY LNA PCR Assays #339306 according to the manufacturer's instructions. Preparation of 384-well PCR plates was conducted on the Opentrons OT-2 liquid-handling robot using a custom protocol designed in Python 3.0. To 7 μL master mix, 3 μL sample was added to each well and mixed five times using the robot. Each plate contained three no-template controls per gene and triplicate inter-plate calibrators to allow for cross-plate comparison (TATAA, Sweden). qPCR was run on the BioRad CFX Opus 384 using the following conditions: initial activation, 2 min, 95 °C; 2-step cycling: denaturation, 10 secs, 95 °C; combined annealing/extension; 60 secs, 56 °C; for a total of 40 cycles. A melt-curve (60-95 °C) for each primer was used to check for non-specific amplification using the sample maximization method. Cq accounting for efficiency was determined from the double derivative method using LinRegPCR.

Cq values were normalized using the geometric mean of three stable reference genes (miR-146a-5p, miR-29b-3p, and miR-126-5p) and a suitability test was calculated to assess suitability (Banack et al., “A microRNA diagnostic biomarker for amyotrophic lateral sclerosis,” Brain Communications, fcae268, pp. 1-15 (2024); Vandesompele et al., “Accurate normalization of real-time quantitative RT-PCR data by geometric averaging of multiple internal control genes,” Genome Biol., Vol. 3 (7), (2002), the disclosures of each of which are incorporated by reference herein in their entireties). Gene-fold expression, 2-(AACq), was calculated from delta-delta Cq (normalized sample Cq minus the mean of normalized control sample Cqs). Fold regulation was calculated using the mean gene fold expression for AD or MCI divided by the mean gene fold expression for control samples. For this analyses values greater than four standard deviations were considered extreme outliers and were eliminated from the analysis for that specific miRNA alone (n=0 for circulating cohort 1; n=6 for circulating cohort 2; and n=8 for circulating cohort 3). Fold regulation was defined as equal to gene fold change when it was greater than one and was calculated as negative one divided by gene fold change when the fold change was less than one.

The data from this analysis was used to compare miRNA from AD and/or MCI subjects to healthy control subjects to assess the fold difference, and those miRNA that exhibited sufficiently consistent differences are provided in Table 4 below. The data also was used to compare AD and MCI subjects (MCI subjects in this instance could be considered a control) to assess the fold differences in miRNA and thereby provide an miRNA fingerprint that could be used to assess whether a subject was, or was at risk of progressing from MCI to AD. The miRNA that showed sufficiently consistent fold changes (e.g., increases or decreases) along with the respective fold changes are provided in Table 4 below:

TABLE 4

| | AD / Control | | | | MCI / Control | | | | Disease Progression from MCI to AD | | | |
|---|---|---|---|---|---|---|---|---|---|---|---|---|
| | Cohort 1 | | Cohort 2 + 3 | | | | Cohort 2 | | | | | |
| | 39 AD / 42 | | 87 AD (62 + 25) / | | Cohort 1 | | 27 MCI (21 + 6) / | | | | Cohort 2 | |
| | Control | | 182 Control | | 39 MCI / 42 | | 182 Control | | Cohort 1 | | 27 MCI (21 + 6) / | |
| | up | down | (82 + 100) | | Control | | (82 + 100) | | 39 MCI 39 AD | | 87 AD (62 + 25) | |
| | regu- | regu- | up | down | up | down | up | down | up | down | up | down |
| miRNA | lated | lated | regulated | regulated | regulated | regulated | regulated | regulated | regulated | regulated | regulated | regulated |
| 151a-3p | 1.99 | | 2.12 | | 2.01 | | 2.40 | | | −1.01 | | −1.13 |
| 151a-5p | 1.23 | | 1.87 | | 2.22 | | 3.38 | | | −1.81 | | −1.81 |
| 146a-5p | 2.22 | | 1.45 | | 1.61 | | 1.70 | | 1.38 | | | −1.18 |
| 4454 | | −4.25 | | −2.31 | | −8.96 | | −7.47 | 2.11 | | 3.23 | |
| 10b-5p | | −3.70 | | −3.33 | | −5.03 | | −11.32 | 1.36 | | 3.40 | |
| 199a-3p | 1.67 | | 1.75 | | 1.79 | | 2.47 | | | −1.07 | | −1.41 |
| 199a-5p | 1.63 | | 2.92 | | 2.21 | | 5.73 | | | −1.35 | | −1.96 |
| 29b-3p | | −2.03 | 1.00 | | | −1.30 | | −1.36 | | −1.56 | 1.36 | |
| 126-5p | | −1.21 | | −1.33 | | −1.29 | | −1.52 | 1.06 | | 1.14 | |
| 150-5p | | −5.74 | | −3.83 | | −6.15 | | −8.77 | 1.07 | | 2.29 | |
| 107 | | −1.24 | 1.75 | | 1.78 | | 3.62 | | | −2.22 | | −2.07 |
| let-7g-5p | | −1.32 | | −13.61 | 1.04 | | | −15.98 | | −1.37 | 1.17 | |
| 1290 | | −7.42 | | −7.86 | | −14.87 | | −24.02 | 2.00 | | 3.06 | |
| 95-3p | | −4.17 | | −3.80 | | −3.85 | | −7.61 | | −1.08 | 2.00 | |
| 375-3p | | −2.11 | | −2.56 | | −1.69 | | −4.11 | | −1.25 | 1.61 | |
| 99a-5p | | −2.55 | | −2.79 | | −3.21 | | −5.58 | 1.26 | | 2.00 | |
| 125b-5p | | −2.52 | | −2.65 | | −2.49 | | −4.22 | | −1.01 | 1.59 | |
| 642a-5p | | −4.76 | | −1.30 | | −6.19 | | −7.25 | 1.30 | | 5.57 | |
| 92a-1-5p | | −4.22 | | −2.61 | | −3.98 | | −3.33 | | −1.06 | 1.28 | |
| 362-5p | | −4.64 | | −17.87 | | −2.60 | | −37.96 | | −1.78 | 2.12 | |
| 342-3p | | −2.48 | | −2.75 | | −2.16 | | −3.24 | | −1.15 | 1.18 | |
| 196a-5p | | −2.89 | | −2.09 | | −1.82 | | −2.70 | | −1.59 | 1.29 | |
| 155-5p | | −2.10 | | −1.74 | | −1.76 | | −1.38 | | −1.20 | | −1.26 |
| 409-3p | 3.20 | | 2.70 | | 1.74 | | 4.17 | | 1.84 | | | −1.55 |
| 744-5p | 2.27 | | 2.20 | | 2.28 | | 3.88 | | 1.00 | | | −1.76 |
| 223-3p | | −1.06 | 2.13 | | 1.34 | | 2.47 | | | −1.42 | | −1.16 |
| 584-5p | 2.90 | | 1.50 | | 1.49 | | 1.44 | | 1.95 | | 1.04 | |
| 340-5p | 2.56 | | | −3.28 | 1.64 | | | −2.62 | 1.56 | | | −1.25 |
| 421 | 2.08 | | 1.44 | | 1.56 | | 1.24 | | 1.33 | | 1.16 | |
| 486-5p | | −1.06 | 1.08 | | | −1.08 | | −2.64 | 1.02 | | 2.84 | |
| 126-3p | | −1.90 | | −1.70 | | −1.44 | | −1.37 | | −1.32 | | −1.24 |
| 320c | 1.16 | | | −1.69 | | −2.22 | | −2.68 | 2.58 | | 1.59 | |
| 320b | 1.21 | | | −1.67 | | −2.03 | | −2.43 | 2.46 | | 1.46 | |
| let-7d-3p | 1.11 | | | −1.45 | | −1.49 | | −1.66 | 1.65 | | 1.14 | |
| 27b-3p | 1.43 | | 1.51 | | 1.43 | | 1.85 | | | −1.01 | | −1.23 |
| 148b-3p | 2.05 | | | −1.09 | 1.48 | | 1.55 | | 1.39 | | | −1.69 |
| 143-3p | 1.04 | | | −22.16 | 1.60 | | | −29.07 | | −1.54 | 1.31 | |
| 326 | 2.10 | | 1.51 | | | −1.03 | 1.61 | | 2.15 | | | −1.06 |
| 335-5p | 2.60 | | 1.01 | | 1.28 | | | −1.22 | 2.03 | | 1.24 | |
| 25-3p | | −1.19 | 1.68 | | 1.09 | | | −1.14 | | −1.29 | 1.91 | |
| 153-3p | | −1.27 | | −1.48 | 1.46 | | | −1.24 | | −1.86 | | −1.19 |
| 19b-3p | | −1.68 | 2.00 | | 1.18 | | 1.33 | | | −1.98 | 1.51 | |
| let-7c-5p | | −1.17 | | −1.14 | 1.04 | | 1.30 | | | −1.21 | | −1.47 |
| Cq-652-3p | no data | | 1.95 | | no data | | 2.75 | | no data | | | −1.41 |
| Cq-376c-3p | no data | | 2.52 | | no data | | 2.86 | | no data | | | −1.14 |
| Cq-154-5p | no data | | 1.61 | | no data | | 1.63 | | no data | | | −1.01 |
| Cq-376a-3p | no data | | | −5.26 | no data | | | −7.97 | no data | | 1.52 | |
| Cq-30d-5p | no data | | 2.10 | | no data | | 2.35 | | no data | | | −1.12 |
| Cq-381-3p | no data | | 2.46 | | no data | | 3.99 | | no data | | | −1.62 |

### Classification Algorithms

The data from Table 4 was used to create a prediction model for the assessment of accuracy, sensitivity, and specificity. The data were subject to a ten-fold cross-validation, multinomial, relaxed fit, glmnet model in R-studio using the several libraries (data.table, glmnet, knitr, and caret), softmax function, deltaCq values from 50 miRNA as the X variable and class identification as the y variable, seedset of 42, mean square error as the measure, z-score data standardization, lasso variable selection, with a convergence threshold of 1e-7, and 1e7 maximum iterations. An algorithm was created, using the techniques described herein, that selected, through a feature selection or variable selection, which of the miRNA examined through qPCR contributed the most to separating AD, MCI, and healthy controls. Using the classification algorithm, a model was created based on the data from hundreds of individual patients with known disease classification. Coefficients were assigned to each of the miRNA based on the relative contribution of each miRNA to the model. Using this model, the combined qPCR data from a single individual can then be compared to the model to determine a disease classification prediction for that individual.

The Cross-Validation glmnet Model in R data is provided in Table 5 below.

TABLE 5

| Class | AD (%) | Control (%) | MCI (%) |
|---|---|---|---|
| Sensitivity | 79 | 91 | 79 |
| Specificity | 90 | 90 | 96 |
| Positive | 78 | 91 | 79 |
| Predictive | | | |
| Value | | | |
| Negative | 91 | 90 | 96 |
| Predictive | | | |
| Value | | | |
| Balanced | 84 | 91 | 88 |
| Accuracy | | | |
| AUC | 80 | 92 | 91 |

Accuracy: 0.85
95% CI: (0.82, 0.89)
P-Value [Acc > NIR]: <2e−16
Kappa: 0.75
Mcnemar's Test P-Value: 0.83

### Example 2—Creating a Predictive Model and System to Diagnose AD and/or MCI

Mild cognitive impairment (MCI) is generally considered a transitional state between normal cognitive aging and Alzheimer's disease (AD). However, the line at which an individual crosses over from MCI to AD is murky, and reversal from MCI back to a healthy state has been calculated in 16% of diagnosed patients (Koepsell T D, Monsell S E, “Reversion from mild cognitive impairment to normal or near-normal cognition: risk factors and prognosis,” Neurology, 2012 Oct. 9; 79(15):1591-8. doi: 10.1212/WNL.0b013e31826e26b7. Epub 2012 Sep. 26. PMID: 23019264; PMCID: PMC3475624). Furthermore, the underlying pathology and risk factors leading to MCI and from MCI to AD is controversial. In any given year, the probability of developing AD after the initial diagnosis of MCI is about 15% (Wang Y, Li M, Haughton D, Kazis LE, “Transition of mild cognitive impairment to Alzheimer's disease: Medications as modifiable risk factors,” PLoS ONE 19(8):e0306270, 2024; https://doi.org/10.1371/journal.pone.0306270).

This example identifies a miRNA signature found in blood plasma that aids in diagnosing and separating MCI and AD, with the aid of a multinomial logistic regression model to create the miRNA signature model and system. Venous blood samples from individuals identified by physicians with MCI (n=92) and AD (n=185), as well as from individuals without known cognitive impairment (n=428) were collected in K2EDTA tubes and centrifuged to separate the blood plasma. Extraction of neural-enriched extra-cellular vesicles was conducted from a subset of blood samples using immunoaffinity, as described in Example 1 above, in U.S. Patent Application Publication No. 2021/0164051, and Banack et al., “An miRNA fingerprint using neural-enriched extracellular vesicles from blood plasma: towards a biomarker for amyotrophic lateral sclerosis/motor neuron disease,” Open Biol., Vol. 10, pp 1-12 (2020), the disclosure of which is incorporated by reference herein in its entirety. Next generation sequencing was performed to identify differentially expressed miRNA between groups (Banack et al. 2020). RNA was then extracted directly from the blood plasma of a large number of individuals. Blood plasma includes RNA circulating in the blood plus RNA found in extracellular vesicles within the blood and the RNA extraction process collects both simultaneously. Following cDNA synthesis, a qPCR analysis was carried out on the 49 miRNA targets listed in Table 4 above as a more precise measurement of miRNA abundance, using the same techniques discussed in Example 1. The raw data from qPCR was normalized using the geometric mean of 3 stable endogenous miRNA to account for RNA loading.

The normalized qPCR data from all 705 individuals were then used to conduct a relaxed fit, no penalty, multinomial logistic regression (glmnet) in R-studio with four libraries: data.table, glmnet, knitr, and caret. The model used a lasso feature selection which kept out-of-fold predictions and included the following parameters: seed set of 42, multinomial family, grouped type, mse measure, Z-score standardization, intercept included, lambda sequence (exp(seq(−4,−1, length.out=1000), 20 nfolds, convergence threshold of 1e-6, 1e7 maximum iterations.

The fit model calculated an AUC score of 0.84, which is a measure of the entire two dimensional area under the Receiver Operation Characteristic curve. The mean standard error was 0.08. Overall accuracy was 0.84 (95% CI: 0.812, 0.8674) and a Kappa score of 0.70. The classification sensitivity, specificity, and model predictive values were high (Table 6).

TABLE 6

| Class | AD (%) | Control (%) | MCI (%) |
|---|---|---|---|
| Sensitivity | 76.2 | 91.8 | 64.1 |
| Specificity | 92.3 | 83 | 96 |
| Positive Predictive | 77.9 | 89.3 | 70.2 |
| Value | | | |
| Negative Predictive | 91.6 | 86.8 | 94.7 |
| Value | | | |
| Prevalence | 26.2 | 60.7 | 13.1 |
| Detection Rate | 20 | 55.7 | 8.4 |
| Detection | 25.7 | 62.4 | 11.9 |
| Prevalence | | | |
| Balanced Accuracy | 84.3 | 87.4 | 80 |

Carrying out the above analysis, 16 miRNA were identified in the model generated to differentiate between AD, MCI, and controls, each with a coefficient identifying the contribution of the miRNA to the model, as shown in Table 7 below. The model used a Lasso feature selection that iteratively determined which miRNA add more to distinguishing the respective disease states and eliminated miRNA that added less. The Lasso feature selection selected the 16 miRNA, which were determined to have the greatest discriminatory power and provided the requisite predictive accuracy. Table 7 provides the general linear model coefficients for Alzheimer's Disease, Mild Cognitive Impairment, and healthy controls.

TABLE 7

| miRNA | Class AD | Class Control | Class MCI |
|---|---|---|---|
| (Intercept) | 7.7818 | −0.3909 | −7.3909 |
| 151a-3p | 0.3876 | 0.7540 | −1.1416 |
| 199a-5p | −0.0529 | 0.1793 | −0.1263 |
| 150-5p | 0.6419 | −0.4377 | −0.2042 |
| 375-3p | 0.1726 | 0.3627 | −0.5352 |
| 99a-5p | −0.3193 | −1.2245 | 1.5439 |
| 642a-5p | −0.4019 | 0.1991 | 0.2028 |
| 92a-1-5p | −0.2409 | 0.0424 | 0.1986 |
| 223-3p | −0.6022 | 0.0793 | 0.5229 |
| 421 | −0.2634 | 0.1374 | 0.1260 |
| 320b | −0.6531 | −0.0350 | 0.6881 |
| 335-5p | 0.1225 | −0.3280 | 0.2055 |
| 153-3p | −0.1706 | 0.1432 | 0.0274 |
| 19b-3p | 0.0911 | 0.5968 | −0.6878 |
| 652-3p | 0.0385 | −0.1141 | 0.0757 |
| 30d-5p | −0.1829 | 0.0420 | 0.1409 |

It is interesting that from the individuals with diagnosed MCI, 16% were predicted to have AD which is consistent with the expected transition rate (Wang et al. 2024). In addition, 20% of diagnosed MCI patients were classified with the control group and this also represents approximately the number that is expected to revert back to normal (Koepsell et al. 2012). The sensitivity of the model, or its ability to predict AD, which is a later disease stage with more severe loss of cognitive function, was higher (76%) and the ability of the model to exclude AD as a diagnosis was 92%. Those skilled in the art recognize that a glm model is a complicated model of logarithmic odds. In the binomial glm model of just (AD and MCI), a one unit change in the miRNA deltaCq multiplies the odds of the person having AD by eB1. Roughly speaking, a higher coefficient adds more to the model than a lower coefficient. A positive coefficient number in the Class AD column for each respective miRNA (e.g., miRNA 151a-3p has a Class AD coefficient of 0.3876) indicates that its presence makes it more likely that the person has AD. In contrast, a negative number (e.g., miRNA 199a-5p has a Class AD coefficient of −0.0529) makes it less likely (when the miRNA is abundant) that the person has AD. The sum of the coefficients in each row for each respective miRNA therefore results in a value of zero. The combined effect of all the miRNA coefficients in each column makes the final prediction.

Carrying out the above analysis, 11 miRNA were identified in the model (using the Lasso feature selection method) that was used to differentiate between AD and MCI, each with a coefficient identifying the contribution of the miRNA to the model, as shown in Table 8 below. Using the same parameters in a multinomial logistic regression model of 185 AD and 92 MCI patients, the model was capable of predicting AD with 90% sensitivity and MCI with 78% sensitivity. The model had an AUC score of 0.93 and mean standard error of 0.11. Table 8 provides the general linear model coefficients for differentiating Alzheimer's Disease from Mild Cognitive Impairment.

TABLE 8

| miRNA | Class AD | Class MCI |
|---|---|---|
| (Intercept) | 1.2565 | −1.2565 |
| 4454 | −0.6488 | 0.6488 |
| 95-3p | 0.3241 | −0.3241 |
| 375-3p | 0.3554 | −0.3554 |
| 642a-5p | −0.3370 | 0.3370 |
| 92a-1-5p | −0.0570 | 0.0570 |
| 196a-5p | 0.2718 | −0.2718 |
| 223-3p | 0.0741 | −0.0741 |
| 421 | −0.1832 | 0.1832 |
| 320b | −0.9178 | 0.9178 |
| 335-5p | 0.0712 | −0.0712 |
| 30d-5p | −0.0781 | 0.0781 |

As expected, this model was more accurate for AD since the underlying disease etiology is less variable than MCI. With the model in place, the combined qPCR data from a single individual can then be compared to the model to determine a disease classification prediction for that individual.

The model, which is a predictive algorithm, may be created using a large number of individuals with a known disease status, and can and likely will be repeatedly updated as more data is fed into the model. Thus, the miRNA and their respective coefficients may change over time (e.g., the identified miRNA and their respective coefficients provided in Tables 7 and 8 above may change over time). But once the algorithm is created as described above in Examples 1 and 2, individuals of unknown disease status can be tested using the model, which will provide a prediction of the likelihood that the individual has or does not have the disease. This can be accomplished by first drawing a blood sample, centrifuging the blood to retrieve the blood plasma fraction, extracting the RNA, creating cDNA, and quantifying the abundance of each of the miRNAs identified in the model using qPCR techniques. The raw cycle quantitation (Cq) numbers can then be normalized by the geomean of three endogenous miRNA as described above, thus generating a deltaCq value for each miRNA, using the techniques discussed in the examples hereinabove (see Banack et al., (2024), and Vandesompele et al., (2002)). These deltaCq values are then entered into the model (e.g., predictive algorithm), and each miRNA value contributes to the prediction of disease state incrementally. In the multinomial model, a positive coefficient means that a one-unit increase in the value for that specific miRNA increases the log-odds of the individual having disease A, and decreases the log-odds of the person having disease B and C, relative to the overall average. When the coefficient is negative, the value for that specific miRNA decreases the log-odds of the individual having disease A. The combination of log-odds for each miRNA in the model determines the final prediction that a person does or does not have the disease. The model adds up the likelihood that the person has AD by adding up each of the log odds ratios. It then generates a prediction number that indicates that the person is predicted to have AD with 98% certainty, for example.

### Discussion of Results

AD and/or MCI patients and the neurologists that diagnose them need an AD and/or MCI diagnostic biomarker. Despite worldwide awareness of AD and/or MCI and a plethora of scientific research, patients are still faced with a significant diagnosis delay. The rapid progression of the diseases increases the need for a quick and accurate diagnostic test from easily obtained bio-samples. Adding a blood-based diagnostic test to confirm suspected AD and/or MCI, as well as suspected progression from MCI to AD and/or AD severity, would help neurologists and benefit patients. The results presented herein have demonstrated a robust, reproducible, sensitive, and accurate ability of a multi-miRNA fingerprint to diagnose AD and/or MCI, to diagnose subjects at risk of developing AD and/or MCI, to diagnose subjects progressing or at risk of progressing from MCI to AD, to test subjects to rule out a future AD and/or MCI diagnosis, and to differentiate between AD, MCI, and other neurological diseases, as well as healthy subjects. This miRNA fingerprint (or biomarker) can be utilized as a simple and rapid secondary measure of disease diagnosis following a neurologist's clinical evaluation. The enhanced diagnostic confidence provided by a biomarker would enable neurologists to diagnosis AD and/or MCI patients, as well as identify subjects at risk of developing AD and/or MCI, or identifying subjects progressing or at risk of progressing from MCI to AD, at an earlier stage. This in turn would benefit patients by providing an opportunity for earlier use of disease modifying AD and/or MCI drugs or other therapies.

In order to qualify as a new diagnostic biomarker with Food and Drug Administration (FDA) approval, four criteria must be met: 1) a needs assessment; 2) context of use; 3) benefit/risk; 4) evidence to support qualification. U.S. Department of Health and Human Services. Biomarker Qualification: Evidentiary Framework Guidance for Industry and Staff 2018, available on the FDA website. The current data evaluates a multi-miRNA AD and/or MCI diagnostic fingerprint providing evidence for benefit/risk through classification algorithms with good discriminatory power. The high sensitivity, specificity, positive predictive value, and negative predictive value suggests that the benefits of the miRNA fingerprint (biomarker) in conjunction with clinical evaluation would outweigh any risk, particularly given the uncertain disease progression of AD and/or MCI.

miRNA represent good candidate biomarker targets due to their regulatory roles in essential cell functions. Many miRNA have been identified as dysregulated in AD and/or MCI patients with on-going research investigating their biomarker potential. Disparity between studies is a concern in miRNA research and can be attributed largely to tissues, methods, sample size, and research focus with a resulting multiplicity of fine detail that can alter the end results between studies. See, e.g., Koshiol J, et al., “Strengths and limitations of laboratory procedures for microRNA detection,” Cancer Epidemiol Biomarkers Prev., 19(4):907-911 (2010); and Shademan B, et al., “MicroRNAs as Targets for Cancer Diagnosis: Interests and Limitations,” Adv Pharm Bull., 13(3):435 (2023). Reproducibility in miRNA studies can be impacted by: a) pre-analytical variables including sample collection and processing procedures; b) RNA isolation, quantification, and handling methods; c) lack of internal quality control measures; d) data acquisition, processing, and statistical analysis variability. Lakkisto P, et al., “Development of circulating microRNA-based biomarkers for medical decision-making: a friendly reminder of what should NOT be done,” Crit Rev Clin Lab Sci., 60(2):141-152 (2023). Furthermore, few studies have been adequately validated using large samples sizes of unique patient cohorts.

The embodiments disclosed herein provide a method to enhance reproducible miRNA discovery intended for clinical applications. This robust AD and/or MCI miRNA fingerprint (“biomarker”) was identified through a series of steps, and then surprisingly discovering that the biomarker can be identified in a simple blood draw and provide a more accurate diagnosis of AD and/or MCI than use of neural-enriched extracellular vesicles. The current cohort of samples represent blood plasma collected from several sources using generally accepted plasma collection protocols but without standardization. These samples are thus more heterogeneous in nature, varied in storage duration, AD and/or MCI stage, and diagnosing neurologist. This diversity in pre-analytical sample sources suggests that the miRNA fingerprint (biomarker) was not sensitive to specific blood plasma collection protocols, an assertion that is consistent with the inventors' prior studies, and increases the likelihood of future reproducibility.

In comparison to all miRNA found to be dysregulated in AD and/or MCI, the multi-miRNA fingerprint identified herein that has been found to be valuable in various combinations, are a small subset of the those identified. However, the multi-miRNA fingerprint has important links to AD and/or MCI biological pathways. The precise function of each miRNA and target pathways related to disease onset and progression are not yet clear. Prior research suggests that the multi-miRNAs affect biological process consistent with neurodegenerative disease affecting such biological processes as oxidative stress, cell viability, motor neuron loss, synaptic transmission, neuron regeneration, neural inflammation, and more. However, many miRNA have multiple targets; including several miRNAs from different pathways can increase the robust nature and potential specificity of the test.

In contrast to known diagnostic methods, the present inventors have discovered the ability of this multi-miRNA fingerprint (biomarker) to diagnose AD and/or MCI with high sensitivity and accuracy. The multi-miRNA fingerprint described herein provides evidence of a strong relationship between this diagnostic biomarker and the gold standard method of clinical disease diagnosis. The analytical performance for NEE is good with important predictive value, while the analytical performance for blood-based detection without exosome isolation is beneficial. The use of independent cohort groups in the machine learning algorithm, with one cohort used for training and another cohort for testing, provides support for the predictive value of the test. The analytical validation of the multi-miRNA fingerprint in this study establishes that the biomarker test is both accurate and reproducible on blood plasma collected using multiple collection protocols. It is not sensitive to variation in standard blood plasma collection, storage, or processing conditions which increases its potential for clinical diagnostic evaluation. Further validation of this biomarker using samples from prospective and longitudinal studies, as well additional neurological controls, would aid its future use and accelerate its introduction into the clinic and clinical trials.

Table 9 below provides the miRNA sequences of the multi-miRNA fingerprint

TABLE 9
miRNA Sequences

| | | Mirabase | |
|---|---|---|---|
| mi-RNA | SEQ | (www.mirabase.org) | |
| name | ID NO: | Accession No. | Sequence |
| 151a-3p | 1 | MIMAT0000757 | CUAGACUGAAGCUCCUUGAGG |
| 151a-5p | 2 | MIMAT0004697 | UCGAGGAGCUCACAGUCUAGU |
| 146a-5p | 3 | MIMAT0000449 | UGAGAACUGAAUUCCAUGGGUU |
| 4454 | 4 | MIMAT0018976 | GGAUCCGAGUCACGGCACCA |
| 10b-5p | 5 | MIMAT0000254 | UACCCUGUAGAACCGAAUUUGUG |
| 199a-3p | 6 | MIMAT0000232 | ACAGUAGUCUGCACAUUGGUUA |
| 199a-5p | 7 | MIMAT0000231 | CCCAGUGUUCAGACUACCUGUUC |
| 29b-3p | 8 | MIMAT0000100 | UAGCACCAUUUGAAAUCAGUGUU |
| 126-5p | 9 | MIMAT0000444 | CAUUAUUACUUUUGGUACGCG |
| 150-5p | 10 | MIMAT0000451 | UCUCCCAACCCUUGUACCAGUG |
| 107 | 11 | MIMAT0000104 | AGCAGCAUUGUACAGGGCUAUCA |
| let-7g-5p | 12 | MIMAT0000414 | UGAGGUAGUAGUUUGUACAGUU |
| 1290 | 13 | MIMAT0005880 | UGGAUUUUUGGAUCAGGGA |
| 95-3p | 14 | MIMAT0000094 | UUCAACGGGUAUUUAUUGAGCA |
| 375-3p | 15 | MIMAT0000728 | UUUGUUCGUUCGGCUCGCGUGA |
| 99a-5p | 16 | MIMAT0000097 | AACCCGUAGAUCCGAUCUUGUG |
| 125b-5p | 17 | MIMAT0000423 | UCCCUGAGACCCUAACUUGUGA |
| 642a-5p | 18 | MIMAT0003312 | GUCCCUCUCCAAAUGUGUCUUG |
| 92a-1-5p | 19 | MIMAT0004507 | AGGUUGGGAUCGGUUGCAAUGCU |
| 362-5p | 20 | MIMAT0000705 | AAUCCUUGGAACCUAGGUGUGAGU |
| 342-3p | 21 | MIMAT0000753 | UCUCACACAGAAAUCGCACCCGU |
| 196a-5p | 22 | MIMAT0000226 | UAGGUAGUUUCAUGUUGUUGGG |
| 155-5p | 23 | MIMAT0000646 | UUAAUGCUAAUCGUGAUAGGGGUU |
| 409-3p | 24 | MIMAT0001639 | GAAUGUUGCUCGGUGAACCCCU |
| 744-5p | 25 | MIMAT0004945 | UGCGGGGCUAGGGCUAACAGCA |
| 223-3p | 26 | MIMAT0000280 | UGUCAGUUUGUCAAAUACCCCA |
| 584-5p | 27 | MIMAT0003249 | UUAUGGUUUGCCUGGGACUGAG |
| 340-5p | 28 | MIMAT0004692 | UUAUAAAGCAAUGAGACUGAUU |
| 421 | 29 | MIMAT0003339 | AUCAACAGACAUUAAUUGGGCGC |
| 486-5p | 30 | MIMAT0002177 | UCCUGUACUGAGCUGCCCCGAG |
| 126-3p | 31 | MIMAT0000445 | UCGUACCGUGAGUAAUAAUGCG |
| 320c | 32 | MIMAT0005793 | AAAAGCUGGGUUGAGAGGGU |
| 320b | 33 | MIMAT0005792 | AAAAGCUGGGUUGAGAGGGCAA |
| let-7d-3p | 34 | MIMAT0004484 | CUAUACGACCUGCUGCCUUUCU |
| 27b-3p | 35 | MIMAT0000419 | UUCACAGUGGCUAAGUUCUGC |
| 148b-3p | 36 | MIMAT0000759 | UCAGUGCAUCACAGAACUUUGU |
| 143-3p | 37 | MIMAT0000435 | UGAGAUGAAGCACUGUAGCUC |
| 326 | 38 | MIMAT0000756 | CCUCUGGGCCCUUCCUCCAG |
| 335-5p | 39 | MIMAT0000765 | UCAAGAGCAAUAACGAAAAAUGU |
| 25-3p | 40 | MIMAT0000081 | CAUUGCACUUGUCUCGGUCUGA |
| 153-3p | 41 | MIMAT0000439 | UUGCAUAGUCACAAAAGUGAUC |
| 19b-3p | 42 | MIMAT0000074 | UGUGCAAAUCCAUGCAAAACUGA |
| let-7c-5p | 43 | MIMAT0000064 | UGAGGUAGUAGGUUGUAUGGUU |
| 652-3p | 44 | MIMAT0003322 | AAUGGCGCCACUAGGGUUGUG |
| 376c-3p | 45 | MIMAT0000720 | AACAUAGAGGAAAUUCCACGU |
| 154-5p | 46 | MIMAT0000452 | UAGGUUAUCCGUGUUGCCUUCG |
| 376a-3p | 47 | MIMAT0000729 | AUCAUAGAGGAAAAUCCACGU |
| 30d-5p | 48 | MIMAT0000245 | UGUAAACAUCCCCGACUGGAAG |
| 381-3p | 49 | MIMAT0000736 | UAUACAAGGGCAAGCUCUCUGU |

**Any or all “T” nucleotide bases in the sequences of the miRNAs shown in Table 8 above may be substituted with a “U” nucleotide base.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The embodiments are generally disclosed herein using affirmative language to describe the numerous features and aspects thereof. The embodiments also specifically include features in which particular subject matter is excluded, in full or in part, such as substances or materials, method steps and conditions, protocols, or procedures. For example, in certain embodiments or aspects of the invention, materials and/or method steps are excluded. Thus, even though the embodiments are generally not expressed herein in terms of what they do not include, aspects that are not expressly excluded are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. Some embodiments of the technology described herein suitably can be practiced in the absence of an element not specifically disclosed herein. Accordingly, in some embodiments the term “comprising” or “comprises” can be replaced with “consisting essentially of” or “consisting of” or grammatical variations thereof. A composition “consisting essentially of” refers to a composition that includes only the active ingredients claimed (e.g., active ingredient (AI) or active pharmaceutical ingredient (API); e.g., L-serine, a salt, metabolic precursor, derivative or conjugate thereof); which composition may include other ingredients such as formulation materials, excipients, additives, carriers, preservatives, diluents, solvents, fillers, salts, buffers, coatings, binders, and lubricating agents; and which composition excludes other APIs not claimed.

The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

1. A method of identifying a subject who has, or is at risk of developing Alzheimer's Disease (AD) comprising:
(a) determining a presence or amount of two or more micro-RNAs (miRNAs) in a subject's circulating blood, wherein the two or more miRNA are selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p, without determining a presence or amount of the miRNAs from neural-derived exosomes; and
(b) determining if the subject has, or is at risk of developing AD according to the presence or amount of the two or more miRNAs in the sample.

2. The method of 1, wherein the method further comprises a method of preventing or treating AD in a subject who has, or is at risk of developing AD the method comprising:
(c) administering a therapeutically effective amount of an AD drug to the subject when the determining of (b) determines that the subject has, or is at risk of developing AD.

3. The method of 2, wherein the AD drug is selected from the group consisting of L-serine, a cholinesterase inhibitor (e.g., donepezil, galantamine, aricept, or rivastigmine), Memantine, Namzaric, an antidepressant (eg. trazodone), an N-methyl D-aspartate (NMDA) antagonist, an omega-3 fatty acid, curcumin, or a curcumin derivative, vitamin E, vitamin B complex or individual vitamin B's; vitamin D, CoQ10, a sleep aid (e.g., zolpidem, eszopiclone or zaleplon), an anti-anxiety drug (e.g., lorazepam, clonazepam, bupropion, Mirtazapine), an anti-convulsant (e.g., sodium valproate, carbamazepine, or oxcarbazepine), an anti-psychotic (e.g., risperidone, quetiapine or olanzapine), carbidopa-levodopa, amantadine, a dopamine agonists (e.g., pramipexole, ropinirole, rotigotine or Apomorphine), a MAO B inhibitor (e.g., selegiline, rasagiline or safinamide), a SSRI drug (eg. Lexapro, Escitalopram),a Catechol O-methyltransferase (COMT) inhibitors (e.g., entacapone or tolcapone), an anticholinerigic (e.g., benztropine or trihexyphenidyl), immunotherapy, anti-amyloid drugs (eg., Lecanemab-irmb, Leqembi, Donanemab, Aducanumab, Aduhelm, Kisunla), anti-Tau drugs (eg., Leuco-methylthioninium-bis(hydromethanesulfonate), Hydromethylthionine mesylate, Salsalate, Gosuranemab, Ponezumab), Prevagen, Gabapentin, Ocuvite, Atenolol, Namenda, anti-hypertension drug (eg., Valsartan, Benazepril, Losartan, Metoprolol), Blood thinners (eg., Eliquis, Xarelto) the like and combinations thereof.

4. The method of 1, wherein the amount of the micro-RNAs determined in (a) is at least 1.0-fold higher or lower than a baseline amount, thereby indicating the subject has, or is a risk of developing, AD.

5. The method of 1, wherein the subject is a human.

6. The method of 1, wherein the subject is asymptomatic for AD.

7. The method of 4, wherein the baseline amount is an average, mean or absolute amount of any one of the miRNAs present in a healthy control subject.

8. The method of 1, further comprising monitoring the progression of AD in the subject, wherein the method is conducted two or more times for the subject.

9. A method of identifying a subject who has, or is at risk of developing Mild Cognitive Impairment (MCI) comprising:
(a) determining a presence or amount of two or more micro-RNAs (miRNAs) in a subject's circulating blood, wherein the two or more miRNA are selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p, without determining a presence or amount of the miRNAs from neural-derived exosomes; and
(b) determining if the subject has, or is at risk of developing MCI according to the presence or amount of the two or more miRNAs in the sample.

10. The method of 9, wherein the method further comprises a method of preventing or treating MCI in a subject who has, or is at risk of developing MCI the method comprising:
(c) administering a therapeutically effective amount of an MCI drug to the subject when the determining of (b) determines that the subject has, or is at risk of developing MCI.

11. The method of 10, wherein the MCI drug is selected from group consisting of L-serine, a cholinesterase inhibitor (e.g., donepezil, galantamine, aricept, or rivastigmine), Memantine, Namzaric, an antidepressant (eg. trazodone), an N-methyl D-aspartate (NMDA) antagonist, an omega-3 fatty acid, curcumin, or a curcumin derivative, vitamin E, vitamin B complex or individual vitamin B's; vitamin D, CoQ10, a sleep aid (e.g., zolpidem, eszopiclone or zaleplon), an anti-anxiety drug (e.g., lorazepam, clonazepam, bupropion, Mirtazapine), an anti-convulsant (e.g., sodium valproate, carbamazepine, or oxcarbazepine), an anti-psychotic (e.g., risperidone, quetiapine or olanzapine), carbidopa-levodopa, amantadine, a dopamine agonists (e.g., pramipexole, ropinirole, rotigotine or Apomorphine), a MAO B inhibitor (e.g., selegiline, rasagiline or safinamide), a SSRI drug (eg. Lexapro, Escitalopram),a Catechol O-methyltransferase (COMT) inhibitors (e.g., entacapone or tolcapone), an anticholinerigic (e.g., benztropine or trihexyphenidyl), immunotherapy, anti-amyloid drugs (eg., Lecanemab-irmb, Leqembi, Donanemab, Aducanumab, Aduhelm, Kisunla), anti-Tau drugs (eg., Leuco-methylthioninium-bis(hydromethanesulfonate), Hydromethylthionine mesylate, Salsalate, Gosuranemab, Ponezumab), Prevagen, Gabapentin, Ocuvite, Atenolol, Namenda, anti-hypertension drug (eg., Valsartan, Benazepril, Losartan, Metoprolol), Blood thinners (eg., Eliquis, Xarelto) the like and combinations thereof.

12. The method of 9, wherein the amount of the micro-RNAs determined in (a) is at least 1.0-fold higher or lower than a baseline amount, thereby indicating the subject has, or is a risk of developing, MCI.

13. The method of 1, wherein the subject is a human.

14. The method of 1, wherein the subject is asymptomatic for MCI.

15. The method of 14, wherein the baseline amount is an average, mean or absolute amount of any one of the miRNAs present in a healthy control subject.

16. The method of claim 9, further comprising monitoring the progression of MCI in the subject, wherein the method is conducted two or more times for the subject.

17. A method of identifying a subject who is, or is at risk of progressing from Mild Cognitive Impairment (MCI) to Alzheimer's Disease, the method comprising:
(a) determining a presence or amount of two or more micro-RNAs (miRNAs) in the circulating blood of a subject previously diagnosed with MCI, wherein the two or more miRNA are selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p, without determining a presence or amount of the miRNAs from neural-derived exosomes; and
(b) determining if the subject has, or is at risk of progressing from MCI to AD according to the presence or amount of the two or more miRNAs in the sample.

18. The method of 17, wherein the amount of the micro-RNAs determined in (a) is at least 1.0-fold higher or lower than a baseline amount determined from a subject previously diagnosed with MCI, thereby indicating the subject has, or is a risk of progressing from MCI to AD.

19. The method of 17, wherein the subject is a human.

20. The method of 18, wherein the baseline amount is an average, mean or absolute amount of any one of the miRNAs present in a subject previously diagnosed with MCI.

21. A method of identifying a subject who is, or is at risk of increased AD disease severity.
(a) determining a presence or amount of two or more micro-RNAs (miRNAs) in the circulating blood of a subject previously diagnosed with MCI, wherein the two or more miRNA are selected from the group consisting of 151a-3p, 151a-5p, 146a-5p, 4454, 10b-5p, 199a-3p, 199a-5p, 26b-3p, 126-5p, 150-5p, 107, let 7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let 7d-3p, 27b-3p, 148b-3p, 143-3p, 326, 335-5p, 25-3p, 153-3p, 196-39, let 7c-5p, 652-3p, 376c-3p, 154-5p, 376a-3p, 30d-5p, and 381-3p, without determining a presence or amount of the miRNAs from neural-derived exosomes;
(b) determining if the subject has, or is at risk of increasing in AD severity according to the presence or amount of the two or more miRNAs in the sample.

22. A system and model useful in identifying a subject who has, or is at risk of developing Alzheimer's Disease (AD) and/or Mild Cognitive Impairment comprising:
a) a database stored in a storage medium, wherein the database comprises dCq values for individuals with a known disease status, a list of miRNA and a coefficient associated with each miRNA, a list of miRNA and a coefficient associated with each miRNA, wherein the list of miRNA is selected from 4 or more miRNA selected from the group consisting of 151a-3p, 151a-5p, 146a-5p, 4454, 10b-5p, 199a-3p, 199a-5p, 29b-3p, 126-5p, 150-5p, 107, let-7g-5p, 1290, 95-3p, 375-3p, 99a-5p, 125b-5p, 642a-5p, 92a-1-5p, 362-5p, 342-3p, 196a-5p, 155-5p, 409-3p, 744-5p, 223-3p, 584-5p, 340-5p, 421, 486-5p, 126-3p, 320c, 320b, let-7d-3p, 27b-3p, 148b-3p, 143-3p, 326, 335-5p, 25-3p, 153-3p, 19b-3p, let-7c-5p, 652-3p, 376c-3p, 154-5p, 376a-3p, 30d-5p, and 381-3p; and
b) software configured to access the database, process qPCR generated quantification data for each miRNA in the list; generate normalized qPCR values for each miRNA in the list, and process the normalized qPCR values for each miRNA by computing and outputting a predicted diagnosis of AD and/or MCI.
